(19) United States
(12) Patent Application Publication
(10) Pub. No.: US 2023/0273223 A1
(43) Pub. Date: Aug. 31, 2023

(54) PANEL OF INFLAMMASOME PROTEINS AS RADIATION BIODOSIMETER

(71) Applicant: THE TRUSTEES OF THE UNIVERSITY OF PENNSYLVANIA, Philadelphia, PA (US)

(72) Inventors: Melpo CHRISTOFIDOU-SOLOMIDOU, Eagleville, PA (US); Shampa Chatterjee, Philadelphia, PA (US); Thais Sielecki-Dzurd, Kennett Square, PA (US); Ralph Pietrofesa, Harrisburg, PA (US)

(73) Assignee: THE TRUSTEES OF THE UNIVERSITY OF PENNSYLVANIA, Philadelphia, PA (US)

(21) Appl. No.: 18/040,641

(22) PCT Filed: Aug. 5, 2021
(86) PCT No.: PCT/US2021/044817

§ 371 Date: Feb. 4, 2023

Related U.S. Application Data
(60) Provisional application No. 63/061,775, filed on Aug. 5, 2020.

(51) Int. Cl. G01N 33/68

(52) U.S. Cl. CPC G01N 33/6893; G01N 2800/40; G01N 2800/7095

(57) ABSTRACT

The present invention relates to a panel of inflammasone biomarkers for detecting exposure to ionizing radiation and/or for determining absorbed dose of ionizing radiation in a subject exposed thereto.

### STATEMENT REGARDING FEDERALLY SPONSORED RESEARCH

This invention was made with U.S. Government support under Federal Award Identifier Number R41ES031778 from the National Institute of Environmental Health Sciences (NIEHS) at the National Institutes of Health (NIH). The U.S. government has certain rights in the invention.

### FIELD OF THE INVENTION

The present invention relates to a panel of inflammasone biomarkers for detecting exposure to ionizing radiation and/or for determining absorbed dose of ionizing radiation in a subject exposed thereto.

### BACKGROUND

Biodosimeters are critical in the event of a large scale radiological emergency (in the form of a nuclear disaster, terrorist attack or radiation exposure of military personnel) where a large population of “exposed” individuals need immediate appropriate medical care or assigned to triage. Rapid, accurate assessment of individual biological dose (i.e., “biodose”) also is relevant to assigning prognosis and informing medical care among military personnel, civilians, and workers in nuclear power plants and other facilities where handling of radioactive materials is required. In addition, biodosimeters can be useful in medical radiology and specifically in Radiation Oncology, Nuclear Medicine, and Diagnostic and Interventional Radiology, as well as in cosmic/galactic radiation exposures of crew members associated with extended lunar or Mars space exploration missions.

Biodosimetry has largely been limited to assessing radiation doses by monitoring clinical symptoms or physical dosimtery badges. Although clinical dosimetric endpoints using the time to onset of vomiting and decline in absolute lymphocyte count provide a rough estimate of biodose, a substantial number of victims will lack vomiting at LD40/60-50/60 doses of 3-4 Gy (false negatives); and it takes 2-4 days for absolute lymphopenia to occur at doses of 2-4 Gy (Waselenko JJK et al., Ann intern Med 2004; 140:1037). Unfortunately, physiologic moieties whose structure or expression patterns are dose-proportional require sophisticated techniques for detection. Additionally, several of these physiologic moieties (genes and proteins) increase only in specific cell types post radiation (lymphocytes etc.) thus necessitating either tissue biopsy or isolation of cells from peripheral blood. These involve invasive procedures and require specialized lab settings and cannot be used to assay radiation exposure on a mass scale.

Current biodosimetry markers that are in use monitor clusters of potential biodosimetry genes or proteins that are radio-sensitive; however such detection procedures involve invasive procedures (specific cells that need to be isolated from peripheral blood), long processing times, specialized facilities and technical expertise for data evaluation. Thus, these assays, even if accurate, are unsuitable for mass casualty screening at the site of the radiological event. During a large-scale radiological emergency, high amounts of radiation exposure is a public health threat. Thus, assessing radiation levels of exposed individuals is critical for determining appropriate medical care and reassuring the non-affected. Accordingly, there is a need for the identification of new, rapid diagnostic biomarkers that are a surrogate for biodose in a mass screening scenario.

### SUMMARY OF THE DISCLOSURE

In an aspect, provided herein is a method of detecting whether a subject has been exposed to ionizing radiation, comprising obtaining a biological sample from the subject and measuring levels of one or more inflammasome biomarkers selected from the group consisting of NOD-like Receptor Protein 3 (NLRP3), Apoptosis-associated speck-like protein containing CARD (ASC), high mobility group box-1 (HMGB1), Interleukins (IL) IL-1β, IL-6, IL-8, soluble intercellular adhesion molecule (sICAM), and soluble vascular cell adhesion molecule (sVCAM) in the biological sample, wherein a change in levels as compared to a non-irradiated control indicates the subject has been exposed to ionizing radiation.

In an aspect, provided herein is a method of determining an absorbed dose of ionizing radiation in a subject who has been or is suspected of having been exposed to ionizing radiation, comprising obtaining a biological sample from the subject; measuring levels of one or more inflammasome biomarkers selected from the group consisting of NLRP3, ASC, HMGB1, IL-1β, IL-6, IL-8, sICAM, and sVCAM in the biological sample to obtain an expression profile; and determining absorbed radiation dose from the expression profile.

In another aspect, provided herein is a method of detecting whether a subject has been exposed to ionizing radiation, comprising: a) obtaining a biological sample from the subject; and b) measuring a protein and/or mRNA level of one or more inflammasome biomarker selected from the group consisting of NLRP3, ASC, HMGB1, IL-1β, IL-6, IL-8, sICAM, and sVCAM in the biological sample, wherein increased levels as compared to a non-irradiated control indicate the subject has been exposed to ionizing radiation.

In an aspect, provided herein is a device configured to perform the method of any of the methods described herein

In an aspect, provided herein is a radiation biodosimeter panel comprising a combination of NLRP3, ASC, HMGB1, IL-1β, IL-6, IL-8, sICAM, and sVCAM inflammasome biomarkers.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an in vitro model to evaluate the effects of radiation on the biodosimeter panel. The vascular network as represented by flow adapted endothelial cells (FAECs). Since the vascular network is exposed to blood flow in vivo, any in vitro model that recapitulates the vasculature requires that endothelial cells be kept under flow conditions. Here, endothelial cells grown on cover slips are inserted into parallel plate chamber (Warner) and attached into a perfusion circuit. The perfusate from the reservoir is drawn by the peristaltic pump into the flow chamber (via a second reservoir or flow damper). The shear stress generated is ˜7 dyn/cm2 which is representative of the shear associated with blood flow in capillaries (Chatterjee, 2018). After 24 h of flow adaptation, these cells are exposed to radiation and assayed for various markers. Scale bar=25 μm.

FIGS. 2A-2B show γH2AX in flow adapted pulmonary microvascular endothelial cells (FAECs) exposed to increasing doses (2-6 Gy) of gamma radiation. γH2AX is a sensitive molecular marker of DNA damage and quantification of individual γH2AX foci by fluorescence microscopy can inform of double stranded breaks (DSB) given that each break has been found to correspond to one γH2AX focus. The presence of γH2AX in radiation exposed FAEC was examined in a gamma-H2AX foci assay as shown (a) by representative fluorescence images and (b) by quantification. Images were acquired by epifluorescence microscopy using a Nikon TMD epifluorescence microscope, equipped a Hamamatsu ORCA-100 digital camera, and Metamorph imaging software (Universal Imaging, West Chester, Pa., USA). Images were acquired at λ_{excitation}=488 nm, λem 500-560, max-530 nm. All images were acquired with the same exposure and acquisition settings. Scale bar=25 microns.

FIGS. 3A-3B show Annexin V in flow adapted pulmonary microvascular endothelial cells (FAECs) exposed to increasing doses (2-6 Gy) of gamma radiation. Annexin V detects apoptotic cells by its ability to bind to phosphatidylserine, a marker of apoptosis or programmed cell death when it is on the outer leaflet of the plasma membrane. Annexin V-FITC shows apoptotic cells (FIG. 3A) by representative fluorescence images and (FIG. 3B) by quantification. Images were acquired by epifluorescence microscopy using a Nikon TMD epifluorescence microscope, equipped a Hamamatsu ORCA-100 digital camera, and Metamorph imaging software (Universal Imaging, West Chester, Pa., USA). Images were acquired at λ_{excitation}=488 nm, λem 500-560, max-530 nm. All images were acquired with the same exposure and acquisition settings. Scale bar=40 microns.

FIGS. 4A-4C show HMGB1 production in control and irradiated FAECs as monitored by immunostaining (upper panel) and ELISA in lower panel. Cells exposed to 2 Gy radiation were fixed and immunostained using anti HMGB1 antibody. B. The supernatant from the cell monolayer was used to assay for HMGB1 using ELISA kits (LSBio Inc).

FIGS. 5A-5C show mRNA levels of HMGB1 (A), NLRP3 (B) and IL-1β (C) in FAECs at 8 and 24 h post exposure to 8 Gy gamma radiation. qPCR was carried out from the cell extracts using appropriate primers.

FIGS. 6A-6B show in vivo effects of total body irradiation (TBI). Mice were exposed to 8 Gy TBI and sacrificed 24 h later. Blood was obtained and plasma isolated by centrifugation. Plasma was assayed for HMGB1 levels. FIG. 6A Upper panel shows quantitative western blotting was carried out using the Odyssey-LiCoR system. Lower panel: Coomassie Stain to show equal protein loading. FIG. 6B shows densitometric analysis of the digital image was carried out using Image J to obtain relative density values in an excel spreadsheet. *p<0.05.

FIGS. 7A-7C show in vivo radiation effects. Mice were exposed to 20 Gy thoracic radiation were sacrificed 24 h later and plasma collected. FIG. 7A shows immunoblotting for HMGB1. Upper panel: Quantitative western blotting was carried out using the Odyssey-LiCoR system. Lower panel: Coomassie Stain to show equal protein loading. FIG. 7B shows densitometric analysis of the digital image was carried out using Image J to obtain relative density values in an excel spreadsheet. In FIG. 7C dot blot shows HMGB1 in plasma of irradiated (IR).

FIG. 8 shows soluble ICAM (sICAM) protein levels in the plasma of mice exposed to 0-20 Gy thoracic radiation. Measurements were made 24 hours post exposure to radiation. Plasma was isolated by centrifugation and assayed for sICAM using ELISA (quantikine) kits.

FIG. 9 shows NLRP3 protein levels in the plasma of mice exposed to 0-20 Gy thoracic radiation. Mice were sacrificed 2 hours post exposure and plasma was isolated by centrifugation and assayed for NLRP3 using ELISA (R and D biosystems) kits.

FIG. 10 shows Interleukin (IL)-6 protein levels in the plasma of mice exposed to 0-20 Gy thoracic radiation. Plasma was isolated by centrifugation and assayed for IL-6 using ELISA (Fisher Sc.) kits.

FIG. 11 shows IL-1β protein levels (marker of NLRP3 inflammasome activation) in the plasma of mice exposed to 0-20 Gy thoracic radiation. Plasma was isolated by centrifugation and assayed for IL-1β using ELISA (R and D biosystems) kits.

FIGS. 12A-12B show NLRP3 levels in plasma of male and female mice 24 hours post total body radiation exposure.

FIGS. 13A-13B shows ASC levels in plasma of male and female mice 24 hours post total body radiation exposure.

FIGS. 14A-14B show IL-6 levels in plasma of male and female mice 24 hours post total body radiation exposure.

FIGS. 15A-15B show IL-1b levels in plasma of male and female mice 24 hours post total body radiation exposure.

FIGS. 16A-16B show IL-8 levels in plasma of male and female mice 24 hours post total body radiation exposure.

FIGS. 17A-17B show HMGB1 levels in plasma of male and female mice 24 hours post total body radiation exposure.

FIGS. 18A-18B show sICAM-1 levels in plasma of male and female mice 24 hours post total body radiation exposure.

FIGS. 19A-19B show sVCAM-1 levels in plasma of male and female mice 24 hours post total body radiation exposure.

FIGS. 20A-20C show the mathematical model building using the findings from the biodosimetry panel of 8 blood proteins identified in a mouse radiation exposure model. Radiation exposure was the independent variable and blood levels of 8 inflammasome proteins were the dependent (outcome) variables (FIG. 20A). For statistical modeling, the blood levels of measured biomarkers are treated as the independent (predictor) variables and radiation exposure is the dependent (outcome) variable (FIG. 20B). The building of a statistical model to determine not only the individual (simple) effects of the separate biomarkers in predicting radiation exposure, but also the combined effect of multiple biomarkers and their interaction (FIG. 20C).

FIGS. 21A-21H show the correlation analysis displaying the linear relationships between individual biomarker (IL-10, IL-6, IL-8, NLRP3, HMGB1, sICAM-1, sVCAM-1, and ASC) levels and radiation exposure. All 8 biomarkers are statistically significantly correlated with radiation exposure with ASC and NLRP3 having the strongest correlation coefficients at 0.7968 and 0.8564, respectively. Correlations between irradiated dose and blood biomarker level were analyzed by Pearson's correlation coefficients.

FIG. 22 shows the predictive power of the 7 biomarker panel with a Pearson correlation coefficient >0.95 for predicted versus actual radiation dose. Results from the stepwise regression analysis model show that the model was capable of radiation dose assessment with the average predicted radiation dose for 0 Gy equal to 0.27 Gy, 5 Gy equal to 5.60 Gy, 10 Gy equal to 11.31 Gy, 15 Gy equal to 13.55 Gy, and 20 Gy equal to 19.23 Gy.

FIGS. 23A-23E show the findings for the selected model using stepwise linear regression against other alternative models that include different numbers of biomarkers (correlations between predicted versus actual radiation dose). FIG. 23A shows the correlation between predicted vs. actual radiation dose using the Full Model with All Predictor Variables. FIG. 23B shows the correlation between predicted vs. actual radiation dose using only predictors with r>0.7. FIG. 23C shows the correlation between predicted vs. actual radiation dose using only predictors with r>0.5. FIG. 23D shows the correlation between predicted vs. actual radiation dose using only predictors with r>0.8. FIG. 23E shows the correlation for only predictors with r>0.85.

### DETAILED DESCRIPTION

The present subject matter may be understood more readily by reference to the following detailed description that forms a part of this disclosure. It is to be understood that this invention is not limited to the specific products, methods, conditions or parameters described and/or shown herein, and that the terminology used is for the purpose of describing particular aspect and embodiments by way of example only and is not intended to be limiting of the claimed invention.

In embodiments, provided herein is a potential biodosimetry test that is highly sensitive, and suitable for rapid testing of blood. In an embodiment, the biodosimetry test detects exposure to radiation in 1 to 15 minutes. In some embodiments, radiation exposure is detected in 1 to 10 minutes. In an embodiment, radiation exposure is detected in 1 to 5 minutes. In some embodiments, radiation exposure is detected in 1 to 3 minutes. In certain embodiments, radiation exposure is detected in 1 minute. In the aftermath of an improvised nuclear device (IND), for example, or a nuclear accident, having rapid and accurate measurements of internal ionizing radiation absorption in exposed persons can inform life-saving medical decisions. Exposure to radiation can be in the form of low-level background or occupational exposure. In addition, it could be from exposure resulting from a radiation disaster incident such as a nuclear plant accident resulting ion radioactive fallout that includes radioactive iodine 131, cesium 137, strontium 90, plutonium, uranium and other possible isotopes. Additionally, it can result from radiological terrorism such as the detonation of a radiation dispersion device (RDD) and the release of radioactive substances. Contamination may be in the form of external radiation exposure, superficial contamination and incorporation of radioactive material. In addition, exposure can be for cancer patients in the context of medical radiology and specifically in Radiation Oncology, Nuclear Medicine, and Diagnostic and Interventional Radiology as well as in cosmic/galactic radiation exposures of crew members associated with extended lunar or Mars space exploration missions.

The biodosimetry test is a dose-dependent measurement of the biological changes caused by ionizing radiation. The test results will assist physicians and first responders in deciding the level, duration, and combination of medical care administered to an individual.

In an embodiment, the present invention relates to a panel of inflammasome proteins that are radiosensitive and are part of the cellular “danger” sensing machinery. These inflammasome proteins also have an ability to be secreted from the tissue and vascular network into the circulation. In an embodiment, the panel includes one or more of the following inflammasomes: NLRP3, its adaptor molecule ASC, its upstream effector HMGB1 (high mobility group box-1), downstream effectors interleukins IL-1β, IL-6, IL-8, and soluble cellular adhesion molecules (sICAM, sVCAM).

Unless otherwise defined herein, scientific, and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease (including, but not limited to, skin burns and acute radiation syndrome (“radiation sickness”) and long-term adverse health effects, such as cancer and cardiovascular disease) or condition (exposure to ionizing radiation of at least 1 Gy). Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “composition,” “formulation”, “composition of compounds,” “compound,” “drug,” “pharmacologically active agent,” “active agent,” “therapeutic,” “therapy,” “treatment,” or “medicament” are used interchangeably herein, as context dictates, to refer to a compound or compounds or composition of matter which, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment with a pharmaceutical composition in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. Any formulations, methods, or treatments described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, leporidae, or porcine. In another embodiment, the subject is mammalian.

Conditions and disorders in a subject for which a particular drug or compound or composition (or combination thereof) is said herein to be “indicated” are not restricted to conditions and disorders for which that drug or compound or composition has been expressly approved by a regulatory authority, but also include other conditions and disorders known or reasonably believed by a physician to be amenable to treatment with that drug or compound or composition or combination thereof.

Abbreviations used herein include, inter alia: ASC: Apoptosis-associated speck-like protein containing CARD; ELISA: enzyme-linked immunosorbent assays; FAEC: flow-adapted endothelial cell; HMGB1: High Mobility Group Box 1; IACUC: Institutional Animal Care and Use Committee; IL-1β: interleukin-IL-1β; IL-6: interleukin-6; IL-8: Interleukin-8; NLRP3: NOD like receptor protein 3; PBS: phosphate-buffered saline; qRT-PCR: quantitative reverse transcriptase polymerase chain reaction; ROS: reactive oxygen species; sICAM: soluble intercellular adhesion molecule; sVCAM: soluble vascular cell adhesion molecule; TBI: total body irradiation.

In an embodiment, provided herein is a method of detecting whether a subject has been exposed to ionizing radiation, comprising obtaining a biological sample from the subject; and measuring levels of one or more inflammasome biomarkers selected from the group consisting of NLRP3, ASC, HMGB1, IL-1β, IL-6, IL-8, sICAM, and sVCAM in the biological sample, wherein a change in levels as compared to a non-irradiated control indicates the subject has been exposed to ionizing radiation. In an embodiment, the level is a protein level. In an embodiment, the level is an mRNA level.

In an embodiment, the present invention relates to a method of detecting whether a subject has been exposed to ionizing radiation, comprising: a) obtaining a biological sample from the subject; and b) measuring a protein and/or mRNA level of one or more inflammasome biomarker selected from the group consisting of NLRP3, ASC, HMGB1, IL-1β, IL-6, IL-8, sICAM, and sVCAM in the biological sample, wherein increased levels as compared to a non-irradiated control indicate the subject has been exposed to ionizing radiation.

In an embodiment, the measuring comprises ELISA, immunoassay, immunostaining and fluorescence, western blot, or dot blot, immunoprecipitation, RT-PCR, nucleic acid hybridization, or a combination thereof. Any other suitable measuring means known to one of ordinary skill in the art can be employed.

In an embodiment, elevated levels of HMGB1 and/or sICAM-1 as compared to the control indicates the subject has been exposed to ionizing radiation. In an embodiment, elevated levels of three or more of the inflammasome biomarkers as compared to the control indicates the subject has been exposed to ionizing radiation.

In an embodiment, the measuring occurs about 8 hours after exposure. In another embodiment, the measuring occurs about 24 hours after exposure. In another embodiment, the measuring occurs about 1 to about 14 days after exposure.

In an embodiment, the biological sample comprises blood. In an embodiment, the sample can be a serum or plasma sample. In an embodiment, the sample can be obtained in a minimally invasive procedure, such as a finger stick. Any biological sample suitable for use in the methods described herein can be employed.

In an embodiment, the method further comprises treating the subject for radiation exposure. Any suitable treatment for treating such exposure can be used, including, without limitation therapy, monitoring, or further assessment. Treatment for radiological exposures, also known as medical countermeasures (“MCM”) for radiological exposures, will depend on the type of exposure (whether internal, requiring decorporation treatment or external). Treatment may involve administration of a therapeutically effective amount of bacteriostatic agents, NSAIDs, immunomodulatory agents, stem cell transplantation, antibiotics and/or other agents, or other appropriate treatment known and understood by one of ordinary skill in the art. In an embodiment, additional MCM for radiological exposures of a subject who has been or is suspected of having been exposed to ionizing radiation of at least 1 Gy, include but are not limited to, administration of a therapeutically effective amount of potassium iodide (for radioactive iodine exposure), Prussian blue (for cesium and thallium exposure), or chelating agents (for plutonium, americium, curium exposure). In certain embodiments, treatment of the subject for radiation exposure comprises administering a therapeutically effective amount of a growth factor, wherein the growth factor is filgrastim/G-CSF (Neupogen) to treat neutropenia; PEGylated filgrastim/PEGylated G-CSF (Neulasta) to treat febrile neutropenia and reduce the chance of infection due to a low white blood cell count; sargramostim/GM-CSF (Leukine) to stimulate the production of white blood cells and decrease the risk of infection in conditions such as cancer, bone marrow transplant, and pre-chemotherapy blood cell collection; fusion protein thrombopoietin (TPO) peptide analog (Nplate/romiplostim) to increase platelet counts; a chemoprotectant, cytoprotectant, or radioprotectant, such as Amifostine/Ethyol to promote the repair of damaged tissue and binding to harmful free radicals released by cells; a thrombopoietin receptor agonist, such as Promacta/Doptelet to treat the symptoms of thrombocytopenia (a lack of platelets in the blood); an angiotensin-converting enzyme inhibitor, such as Capoten/Vasotec/Prinivil/Ramipril; Palifermin to treat oral mucositis in subjects with hematopoietic stem cell transplantation for malignancies, Erythropoietin to increase red blood cell production, including anemia related to kidney dysfunction; IL-3 to stimulate myelopoiesis; IL-11/Oprelvekin to treat thrombocytopenia[a statin to reduce cardiovascular events in cancer patients; a hemorrheologic agent, such as Pentoxifylline, to help blood flow more easily through narrowed arteries; Xigris to treat severe sepsis; immunostimulatory TLR9 ligands, CpG-ODN, to induce a strong humoral immune response; a gold salt, such as Auranofin/Ridaura, to treat painful/tender and swollen joints; Diclofenac sodium to reduce pain and swelling (inflammation), Metformin to increase the radiosensitivity of cancer cells; Surfaxin to treat or prevent respiratory distress syndrome (RDS); an anti-parasitic agent, such as Diethylcarbamazine citrate; a hematopoietic stem cell mobilizer, such as Mozobil, to helps bone marrow release stem cells into the bloodstream for collection and transplantation back into the body; Silverlon to treat blister injuries and/or as a wound/burn dressing, and/or Ciprofloxacin to treat severe Gram-negative bacterial infections. In an embodiment, the MCM for radiological exposures of a subject who has been or is suspected of having been exposed to ionizing radiation of at least 1 Gy, comprises administration of a therapeutically effective amount of aspirin to mitigate the risk of/reduce cancer incidence, such as background colon/colorectal cancer, and mortality rates of crew members exposed to ionizing particles in Low Earth Orbit and deep space missions, and administration of warfarin to subjects exposed to space radiation to decrease stomach, bladder, brain, prostate, and lung cancer incidence.

In an embodiment, the subject is human. In an embodiment, the subject has been or is suspected of having been exposed to ionizing radiation of at least 1 Gy. In an embodiment, the subject has been or is suspected of having been exposed to ionizing radiation of at least 2 Gy.

In another embodiment, provided herein is a method of determining an absorbed dose of ionizing radiation in a subject who has been or is suspected of having been exposed to ionizing radiation, comprising obtaining a biological sample from the subject; measuring levels of one or more inflammasome biomarkers selected from the group consisting of NLRP3, ASC, HMGB1, IL-1β, IL-6, IL-8, sICAM, and sVCAM in the biological sample to obtain an expression profile; and determining absorbed radiation dose from the expression profile. In an embodiment, determining absorbed radiation dose from the expression profile is based on a mathematical algorithm. In an embodiment, the algorithm comprises performing a correlation analysis to determine linear relationships between individual biomarker levels and radiation exposure, and to identify candidate biomarkers to include in the dose prediction model. In an embodiment, any biomarker with a significant univariate test is selected as a candidate for multivariate analysis. In an embodiment, the algorithm comprises selecting individual biomarkers to develop a quantitative, continuous radiation dose prediction model using multiple linear regression and stepwise regression procedures, which will allow for entering and removing biomarker variables into and from the model, along with observing the effect of one predictor variable by holding the other predictor variables constant. In an embodiment, a simple linear regression analysis is first performed to determine if there is a relationship between biomarker levels and radiation exposure and to describe the strength and direction of the relationship; results from the regression analysis describe the predictive value of each individual biomarker on radiation exposure. In an embodiment, all 8 inflammasome biomarkers, NLRP3, ASC, HMGB1, IL-1β, IL-6, IL-8, sICAM, and sVCAM, are statistically significantly correlated with radiation exposure. In an embodiment, inflammasome biomarkers ASC and NLRP3 have the strongest correlation coefficients, 0.7968 and 0.8564, respectively. In an embodiment, all 8 biomarkers, individually, are predictive of radiation dose and candidates for inclusion in the dose prediction model. In an embodiment, a stepwise linear regression analysis is performed to generate a predictive model with the most informative features, while minimizing overfitting. In an embodiment, the stepwise regression analysis model demonstrates that the model was capable of radiation dose assessment with the average predicted radiation dose for 0 Gy equal to 0.27 Gy, 5 Gy equal to 5.60 Gy, 10 Gy equal to 11.31 Gy, 15 Gy equal to 13.55 Gy, and 20 Gy equal to 19.23 Gy. In an embodiment, the dose prediction model has several advantages, such as (1) estimating radiation dose on a continuous scale using a single model that incorporates all data across multiple serum biomarkers, (2) having the ability to estimate specific radiation doses, beyond the doses included in the design of the study used to generate the model, and (3) being able to utilize a flexible and efficient, single radiation dose prediction model, to accurately identify individuals following a mass exposure event.

In an embodiment, the level is a protein level. In an embodiment, the level is an mRNA level.

In an embodiment, the measuring comprises ELISA, immunoassay, western blot, or dot blot, immunoprecipitation, RT-PCR, nucleic acid hybridization, or a combination thereof. Any other suitable measuring means known to one of ordinary skill in the art can be employed.

In an embodiment, the expression profile comprises a dose dependent increase in one or more levels of NLRP3, ASC, HMGB1, IL-1β, IL-6, IL-8, sICAM, and sVCAM HMGB1 and/or sICAM-1.

In an embodiment, the expression profile comprises a dose dependent increase in one or more levels of NLRP3, IL-1β, IL-6, and/or sICAM.

In an embodiment, the measuring occurs about 8 hours after exposure. In another embodiment, the measuring occurs about 24 hours after exposure. In another embodiment, the measuring occurs about 1 to about 14 days after exposure.

In an embodiment, the biological sample comprises blood. In an embodiment, the sample can be a serum or plasma sample. In an embodiment, the sample can be obtained in a minimally invasive procedure, such as a finger stick. Any biological sample suitable for use in the methods described herein can be employed.

In an embodiment, the method further comprises treating the subject for radiation exposure. Any suitable treatment for treating such exposure can be used, including, without limitation therapy, monitoring, or further assessment. Treatment will depend on the type of exposure (whether internal, requiring decorporation treatment or external). Treatment may involve bacteriostatic agents, NSAIDs, immunomodulatory agents, stem cell transplantation, antibiotics and/or other agents, or other appropriate treatment known and understood by one of ordinary skill in the art. In an embodiment, the subject is human. In an embodiment, the subject has been or is suspected of having been exposed to ionizing radiation of at least 1 Gy. In an embodiment, the subject has been or is suspected of having been exposed to ionizing radiation of at least 2 Gy.

In an embodiment, the subject has been or is suspected of having been exposed to ionizing radiation of at least 5 Gy. In an embodiment, the subject has been or is suspected of having been exposed to ionizing radiation of at least 10 Gy. In an embodiment, the subject has been or is suspected of having been exposed to ionizing radiation of at least 15 Gy. In an embodiment, the subject has been or is suspected of having been exposed to ionizing radiation of at least 20 Gy.

In another embodiment, provided herein is a device configured to perform any method described herein. In an embodiment, the device comprises a platform configured to rapidly detect an inflammasome protein or mRNA from a blood drop sample. In an embodiment, the platform is a portable miniature platform.

In another embodiment, provided herein is a radiation biodosimeter panel comprising a combination of NLRP3, ASC, HMGB1, IL-1β, IL-6, IL-8, sICAM, and sVCAM inflammasome biomarkers.

This and other aspects of the present invention are further illustrated by the following non-limiting examples.

### EXAMPLES

### Example 1

A panel of inflammasome proteins were evaluated that have been reported to be radiosensitive and are also part of the cellular “danger’ sensing machinery. These proteins were chosen judiciously based on their radiosensitivity and their ability to be secreted from the tissue and vascular network into the circulation. These 8 inflammasome proteins are NLRP3, its adaptor molecule ASC, its upstream effector HMGB1 (high mobility group box-1), downstream effectors interleukins IL-1β, IL-6, IL-8, soluble cellular adhesion molecule (sICAM, sVCAM).

Methods: We used flow-adapted mouse endothelial cell model exposed to radiation to test secreted levels of these biomarkers. We also used a murine model of total body gamma irradiation (TBI) exposure (0-20 Gy) to determine plasma levels of NLRP3, HMGB1, sICAM (either protein or mRNA levels) 24 h post exposure, as this is the time point within which first responders typically screen affected individuals for triage.

Results: Cell exposure to just 2 Gy gamma radiation induced a time-dependent increase of HMGB1 protein and mRNA levels in cells when evaluated at 8 and 24 hours post exposure. Mice exposed to 2 to 20 Gy TBI showed a dose dependent increase in plasma NLRP3, IL-1β and IL-6 which was significant as compared to untreated controls (as determined by ELISA). In separate experiments, male and female mice were exposed to 0-20 Gy TBI and plasma isolated and processed using western immunoblots and dot blots followed by densitometric analysis which indicated a 5-fold increase of HMGB1. ELISA evaluation of the plasmas indicated a 2-fold increase of soluble ICAM-1 as compared to non-irradiated controls.

Conclusion: We identified a panel of radiation-sensitive biomarkers that can be detected in biological fluids (cell supernatant and murine plasma from male and female mice) following exposure to increasing doses of radiation. These findings will form the basis of our eventual goal of a portable miniature platform that will allow rapid detection of inflammasome protein using a blood drop sample. Such a device for mass casualty screening whereby affected individuals (yes/no. i.e., irradiated/non-irradiated and level of exposure) can be detected in a single easy and rapid test (antibody-antigen reaction) would be an ideal biodosimeter.

Our aim is to identify radiosensitive blood based biomarkers that are detectable in a drop of blood obtained via a minimally-invasive manner by a rapid, yet lab-quality sensitivity assay for mass casualty screening on site. The NLRP3 inflammasome, a well-characterized signaling platform that upon activation drives cell death and tissue injury, is reported to be activated upon radiation exposure. Activation of NLRP3 occurs via the damage associated molecular pattern (DAMP) protein HMGB1 (high mobility group box-1). The HMGB1-NLRP3 axis in turn, activates interleukin IL-1β that drives cell death and is detectable in peripheral blood. Studies in non-human primates have shown that serum amyloid A (SAA), interleukin (IL)-6 and -18, FMS-like tyrosine kinase 3 ligand (FTL3L), and CRP (C—reactive protein) also show an increase in plasma post-radiation exposure.

Acute exposure to ionizing radiation in vivo induces a systemic inflammatory response syndrome (SIRS) that is characterized by the production of pro-inflammatory signals, including IL-6, IL-1, TGF-β and TNF-α. Among atomic bomb survivors, a TH-1 type response was observed with elevations of circulating levels of IFN-Y and ESR (Hayashi T et al., Am J Med 2005; 118:83), and among irradiated mice, thymocytes were induced to produce elevated levels of IL-1β and TGF-β (Gao H et al., J Radiat Res 2018; 59:395). Here, we exploit the NLRP3 inflammasome, whose assembly leads to caspase-1-mediated activation of the IL-1β family of cytokines (Mangan MSJ et al. Nature Rev Drug Discov 2018; 17:588).

Presented herein is pilot data confirming the response of the HMGB1-NLRP3 axis to radiation exposure. When mice were exposed to thoracic (TI) or total body radiation (TBI), plasma HMGB1 and NLRP3 increased several fold, at 24 h post exposure. The period of 24 h corresponds to a time post radiation disaster when first responders decide on the triage/subsequent treatment. SAA, IL-6, IL-18, CRP and FTL3L though independent of the HMGB1-NLRP3 pathway, have been reported to be radiation responsive. However, this panel of 8 proteins has not been evaluated as a biodosimeter. We thus, hypothesize that a panel of 8 proteins comprising of HMGB1, NLRP3, ASC, IL-1β, IL-6, IL-8, sICAM, sVCAM can be used as a radiation biodosimeter. This panel was judiciously selected based on the radiation sensitivity of all these biomarkers (pilot and published data) as well as the association of some of these with the inflammasome pathway which is emerging as a major target of radiation. We posit that elevated levels of this panel of proteins will be radiation dose-dependent. We will validate our hypothesis using an in vivo model of thoracic radiation-exposed mice as well as in human blood from radiotherapy cancer patients with thoracic malignancies.

Inflammasome and associated proteins of the inflammasome pathway (NLRP3, upstream effector HMGB1, downstream effectors IL1-β, IL-6 and IL-8, NLRP3 adaptor protein ASC, as well as adhesion molecules sICAM and sVCAMs) are “inflammation and injury” biomarkers in peripheral blood. Our pilot studies revealed elevated serum levels of HMGB1, sICAM-1 with radiation exposure. Elsewhere too, increased levels of these proteins have been observed in cells (such as lymphocytes, macrophages) in vitro post radiation exposure.

We thus evaluate this panel of 8 inflammasome proteins as a radiation biodosimeter in endothelial cells (EC) in vitro. ECs are a good cell model to evaluate radiation-induced changes for two reasons: 1) ECs represent the vascular network which as a unifying scaffold plays a role in both capturing and transmitting of radiation and radiation induced changes throughout the body, and 2) the ECs lining the blood vessels are in direct contact with the circulation and thus, proteins shed or secreted by EC could be detected from blood. Additionally, radiation-inflammasome relationships in ECs in vitro will also allow validation of medical countermeasure agents.

Using the same in vitro cell model of vascular networks, as described in Chatterjee, Christofidou-Solomidou and Sielecki, et al., Int. J. Mol. Sci. 2019, 20, 176; doi:10.3390/ijms20010176, which is incorporated herein by reference in its entirety, showed that the NLRP3 inflammasome is activated in response to space-relevant ionizing radiation exposure.

Materials and Methods

2.1. Cells and Culture Media

Isolation and culture of pulmonary microvascular endothelial cells have been described previously. Briefly, endothelial cells were grown in Dulbecco's low glucose modified Eagle's medium supplemented with 10% fetal bovine serum (FBS), nonessential amino acids, and penicillin/streptomycin. Endothelial cells were maintained under static culture conditions for several passages before being subjected to flow.

2.2. Exposure of Cells to Shear Stress

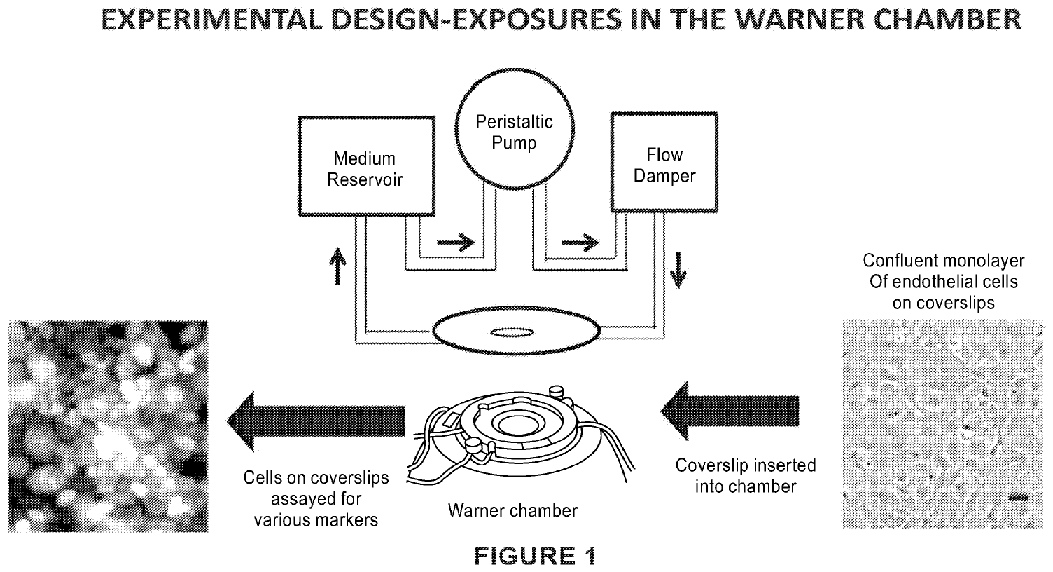

Cells were seeded at 8,000 cells/cm² in medium comprised of low glucose DMEM supplemented with 10% FBS and essential amino acids. The cells reached confluency after 24 h. Cells were lightly washed with medium without FBS and fitted into the chamber slot. A parallel plate confocal imaging chamber (Warner Instruments, LLC, Hamden, Conn., USA) was used to adapt endothelial cells to flow on coverslips as reported earlier. The chamber consisted of two metal circular plates that encased silicone precut gaskets. This created a hollow slot in the center that is fitted with a coverslip containing cells to create a rectangular flow channel (125 μm high, 2 cm wide, and 2 cm long). Inlet and outlet ports on the steel plate were connected to a pump and a dual reservoir to facilitate flow of medium in a pulsatile manner (to recreate the cardiac rhythm) into the system (see FIG. 1: shows a flow system used to recreate the vascular network in vitro). Cells were grown in gelatin-coated cell culture dishes until confluence after which these cells were trypsinized and replated on coverslips pre-coated with fibronectin. Both gelatin and fibronectin serve to replicate the basement matrix in vivo. The basement matrix is the interface between the endothelial cells and adjacent tissue. For gelatin coating, a minimum volume of 1% gelatin was added to the cell culture dishes for 300 min at 37° C. after which cells were plated. Once cells were confluent these were removed and allowed to grow on glass coverslips. To facilitate better adhesion under flow, these coverslips were coated with fibronectin (1 mg/ml in a dilution ratio of 1:100) for 45 min at room temperature, after which excess fibronectin was removed by aspiration. Pulmonary microvascular endothelial cells grown to confluency on fibronectin coated glass coverslips (2 cm×2 cm) were then fitted into the chamber slot, where plastic coverslips were used to seal the flow chambers. With this apparatus, cells were subjected to shear stress for 24 h at 7 dyn/cm².

2.3. Radiation Exposure of FAEC

Flow-adapted endothelial cells were exposed to gamma radiation with a Shepherd Mark 1 ¹³⁷Cs irradiator delivering a dose of 1.0 Gy/minute.

2.4. Determination of ICAM-1 and NLRP3 Expression in FAEC

Levels of ICAM-1 and NLRP3 were determined using an anti-ICAM mouse-monoclonal antibody at 1:250 (ThermoFisher Scientific, Waltham, Mass., USA) and a rabbit polyclonal anti-NLRP3 antibody at 1:200 (R&D Systems, Minneapolis, Minn., USA). Secondary antibodies tagged to fluorescent Alexa 488 (green) were used at 1:200 (ThermoFisher Scientific, Waltham, Mass., USA). Flow-adapted endothelial cells were exposed to gamma radiation and fixed with 4% paraformaldehyde 24 h post radiation exposure and kept at 4° C. Cells were permeabilized and immunostained for ICAM-1 and NLRP3 by using anti-ICAM-1 and anti-NLRP3 antibodies. After washing cells several times and labeling with secondary antibodies, slides were dried and imaged. Images were acquired at 500 ms exposure on a Nikon TMD epifluorescence microscope (Nikon Diaphot TMD, Melville, N.Y., USA), equipped with a Hamamatsu ORCA-100 digital camera (Hamamatsu Photonics K. K., Hamamatsu City, Japan) and MetaMorph imaging software (Molecular Devices, Downington, Pa., USA). The green fluorescent signal in these images represents the amount of ICAM-1 or NLRP3 in these FAECs. The intensity of the fluorescent signal was quantified by integrating the fluorescence of all cells within the entire field and normalizing to the equal field area using Metamorph Imaging Software (Molecular Devices, Downington, Pa., USA) and ImageJ software (Fiji Version, National Institutes of Health, Bethesda, Md., USA). The background was subtracted to obtain “corrected” intensity values. All fluorescence images were acquired at the same exposure and offset acquisition settings. At least 6 fields were imaged and analyzed per condition/treatment and data from 3-4 independent or separate experiments were averaged to obtain the final results. Scale bar=20 m.

2.5. DNA Damage and Apoptosis in FAEC with Radiation:

FAECs exposed to radiation were evaluated for DNA Damage by monitoring γH₂AX expression by using FITC labeled anti-1H2AX followed by microscopic imaging. In separate experiments, FAECs were assessed for cell death (apoptosis) by Annexin V staining of the live cells followed by fixation and imaging.

2.6. RNA Isolation and Gene Expression Analysis

Total RNA was isolated from flow-adapted endothelial cells using a commercially available kit, RNeasy Plus Mini Kit, supplied by Qiagen (Valencia, Calif.), as previously described. Total RNA concentrations and 260/280 ratios were determined using a NanoDrop 2000 apparatus (ThermoFisher Scientific, Waltham, Mass.). Reverse transcription of RNA to cDNA (1.8 μg of total RNA) was then performed on a Veriti® Thermal Cycler using the high capacity RNA to cDNA kit supplied by Applied Biosystems followed by Quantitative Polymerase Chain Reaction (qPCR) analysis using TaqMan® Probe-Based Gene Expression Assays supplied by Applied Biosystems, Life Technologies (Carlsbad, Calif.). Individual TaqMan gene expression assays were selected for heme oxygenase-1 (HO-1), NADPH: quinone oxidoreductase-1 (NQO1), and glutathione S-transferase Mu 1 (GSTM1). Quantitative real-time PCR was performed using 50 ng of cDNA per reaction well on a StepOnePlus™ Real-Time PCR System (Applied Biosystems, Life Technologies, Carlsbad, Calif.). Gene expression data were normalized to both 18S rRNA (FIG. 1) and GAPDH (data not shown) housekeeping genes.

2.7. Dot Blot Analysis

Mouse plasma obtained pre and post thoracic irradiation was evaluated for HMGB1 by dot blotting. This technique for detecting, analyzing, and identifying proteins is similar to the immunoblotting technique but differs in that protein samples are not separated electrophoretically but are spotted through circular templates directly onto the membrane. After absoption on the membrane, the blots are assessed for HMGB1 expression by the use of mouse anti HMG1 and secondary antibody conjugated with HRP.

2.8. Mice

Mice were obtained from Charles River (Wilmington, Mass.) under animal protocols approved by the Institutional Animal Care and Use Committee (IACUC) of the University of Pennsylvania. Animals were housed in conventional cages under standardized conditions with controlled temperature and humidity and a 12:12-hour day-night light cycle. Animals had access to water and standard mouse chow ad libitum. Our kinetic studies and proof of concept studies both used female C57BL/6 mice (n=5 mice per group unless otherwise stated).

2.9. Mouse Irradiation

Radiation Procedure

The Small Animal Radiation Research Platform (SARRP), (Xstrahl, Camberley, United Kingdom) was used to irradiate animals using a custom-made beam collimator. This system uses a Varian model NDI-225-22 kV x-ray tube mounted on a gantry that rotates between 0 and 120 degrees. The custom collimator creates a 12.5 cm circular field with well-defined borders and with animals arranged in a circular, “head in” arrangement using a single central shield which provides uniform irradiation to the thoracic portion of multiple mice simultaneously. This set-up consists of a single, anterior 225 kV, 15 mA x-ray beam with a 0.15 mm Cu at an SSD of 35 cm that is designed to accurately reproduce the internal radiation dose distribution in mice that were used in previous studies. The dosimetry and shielding of this system has been tested extensively. The dose of radiation (0-20 Gy) is a single fraction delivered either via single AP (anterior-posterior) approach or as total body radiation (TBI) exposures.

2.10. Determination of Plasma HMGB1 Levels

Levels of the proinflammatory cytokine High Mobility Group Box 1 (HMGB1) which is released in response to inflammasome activation by a toxicant, were determined in plasma after 24 hours of challenge as described previously. For this, we used enzyme-linked immunosorbent assays (ELISA) using a commercially available ELISA kit (Chondrex Inc., Redmond, Wash.). Samples were run undiluted in triplicate, and assays were performed according to manufacturer's instructions. Levels of HMGB1 released into the BALf are reported as nanograms per milliliter (ng/ml).

2.11. Statistical Analysis

All data were analyzed using two-way analysis of variance (ANOVA). Post-tests (Tukey's multiple comparisons tests) were conducted analyzing significant differences between radiation exposure groups (non-irradiated versus irradiated). Statistically significant differences were determined using GraphPad Prism version 6.00 for Windows, GraphPad Software, La Jolla Calif. USA, www.graphpad.com. Results are reported as the mean±the standard error of the mean (SEM). Levels of target gene mRNA are reported as the mean fold change from FAECs exposed to 0 Gy radiation. Statistically significant differences were determined at p-value of 0.05. # shown in figures indicate significant differences between radiation exposure groups (non-irradiated versus irradiated) (#=p<0.05, ##=p<0.01, ###=p<0.001 and ####=p<0.0001).

Results

In this study it was demonstrated that exposure to gamma radiation leads to increase in time and dose dependent increase in several inflammation markers in the cell model. Additionally, we also observed a time and dose dependent DNA damage and apoptosis in FAEC. We further expanded our studies to murine plasma.

3.1. Gamma Radiation of Flow-Adapted Murine Endothelial Cells Induces Time- and Dose-Dependent Oxidative DNA Damage.

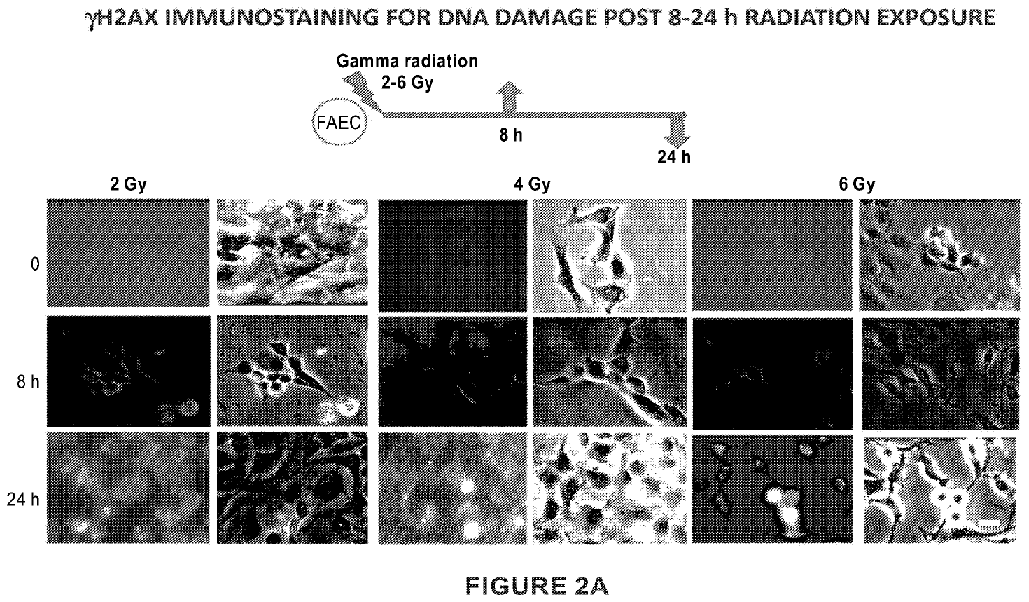

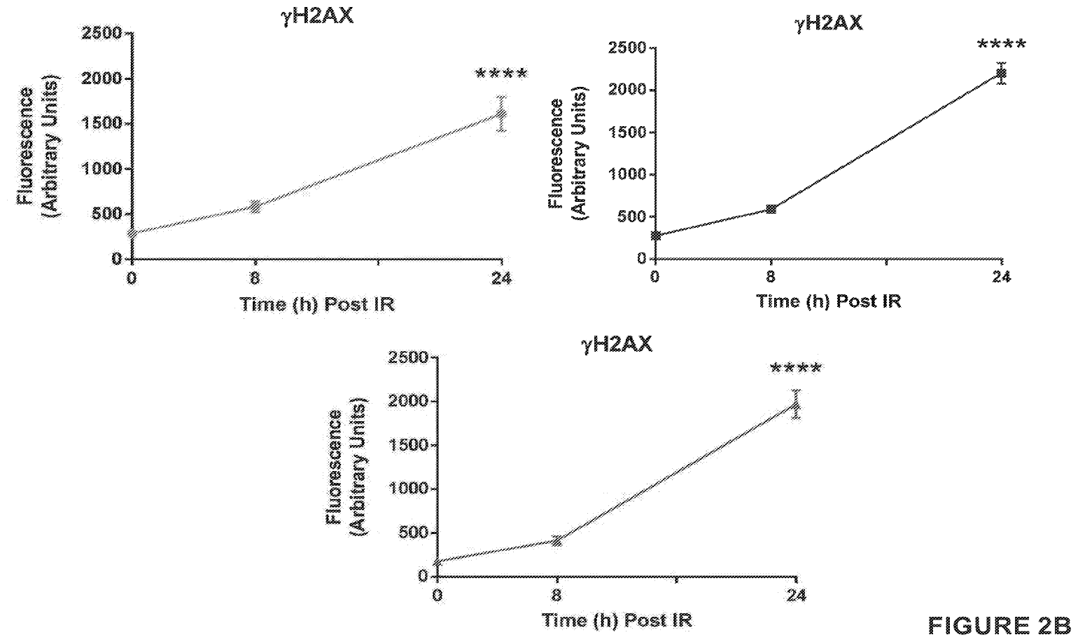

Flow adapted endothelial cells (FAEC) show a dose (0-6 Gy) and time dependent (0, 8, 24 h) increase in DNA damage as observed by γH2AX expression (FIG. 2).

3.2. Gamma Radiation of Flow-Adapted Murine Endothelial Cells Induces Time- and Dose-Dependent Apoptotic Death.

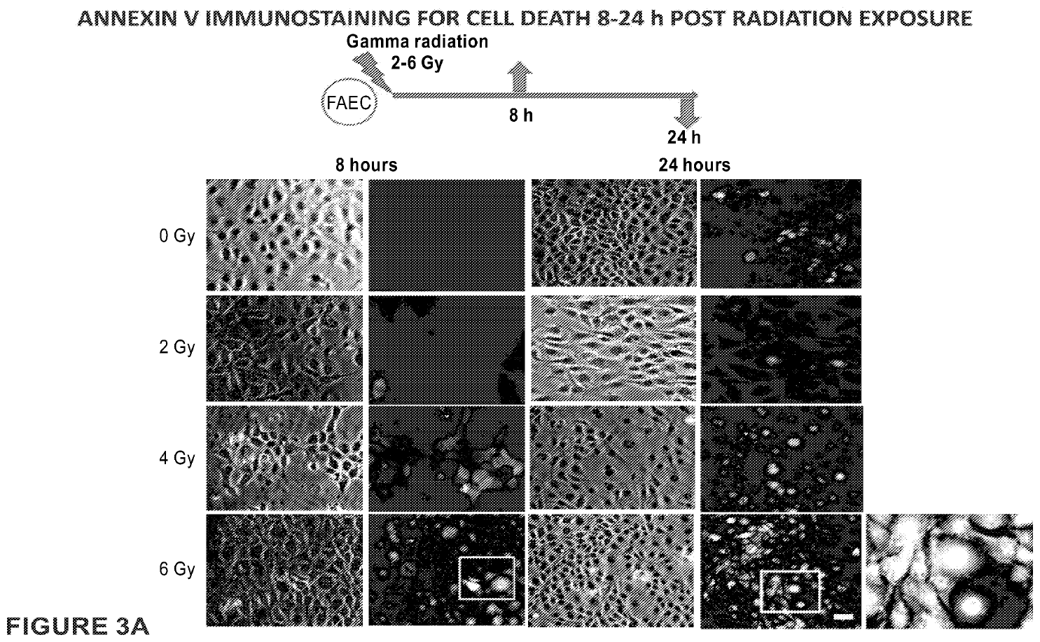

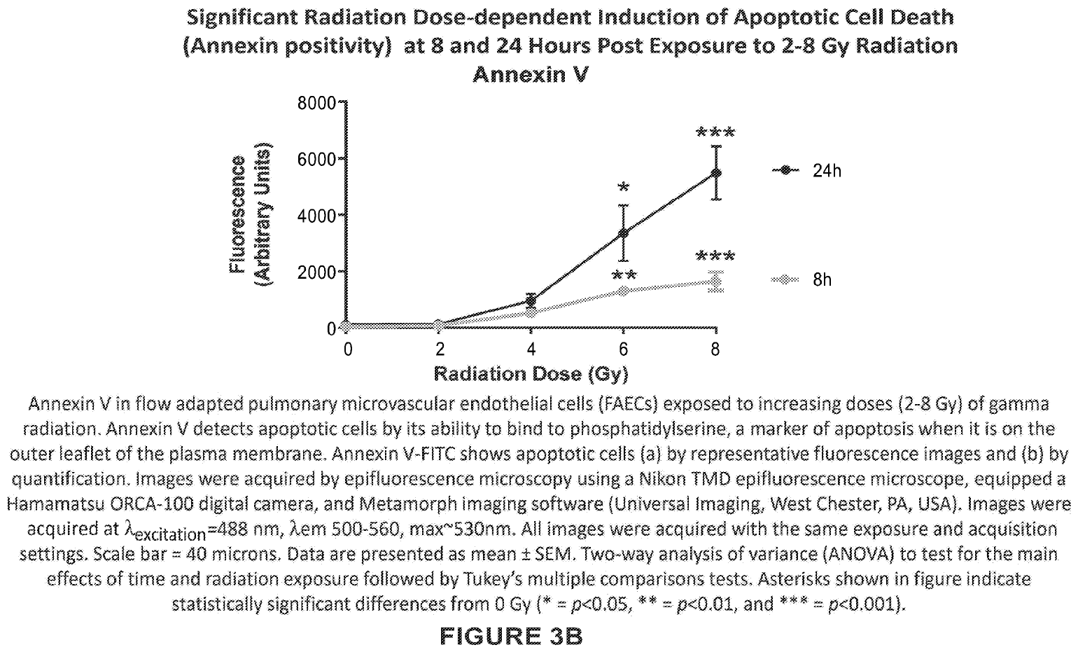

FAECs exposed to radiation (0-6 Gy) showed that apoptosis occurred in a dose and time dependent manner (FIGS. 3A, 3B).

3.3. Gamma Radiation of Flow-Adapted Murine Endothelial Cells Induces Time-Dependent Secretion of Inflammasome Proteins as Well as Induction of Expression.

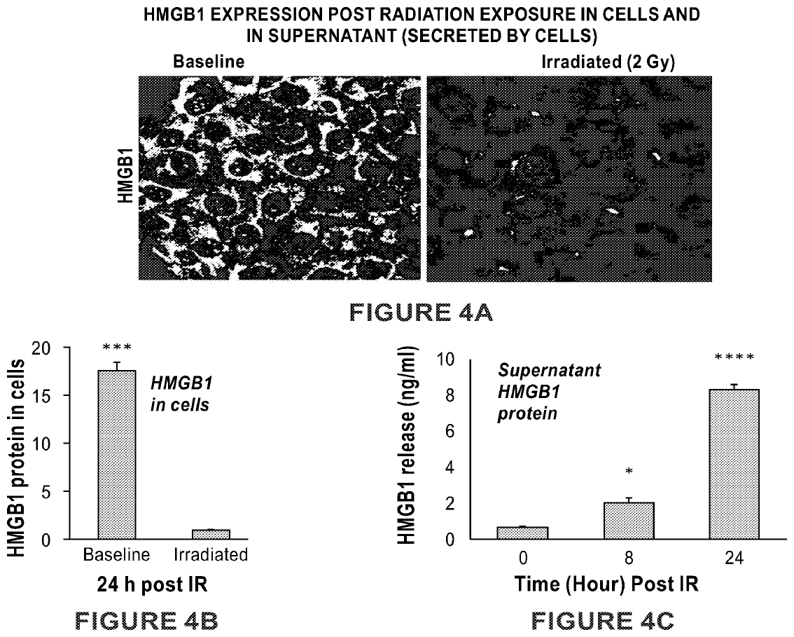

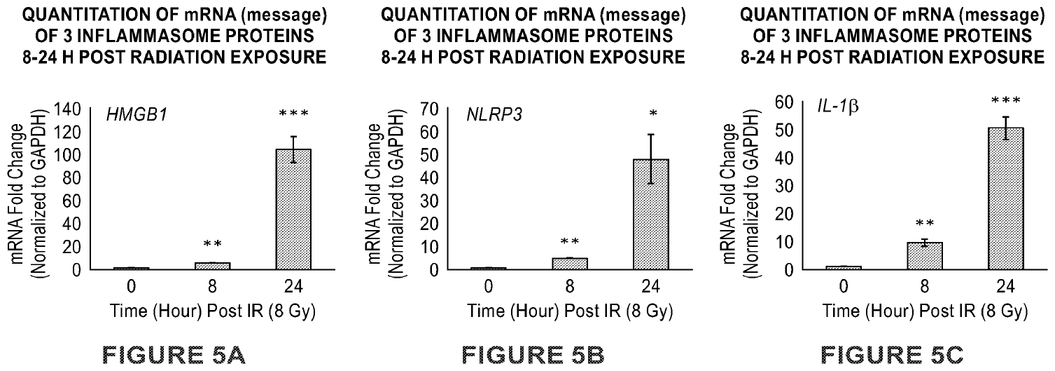

Flow adapted endothelial cells exposed to 2 Gy gamma radiation (FIG. 4A) showed a significant decrease in cellular levels of HMGB1, while secreted levels detected in supernatant, increased in a time-dependent manner (FIG. 4B). Gene expression evaluation using qRT-PCR of FAEC exposed to 8 Gy gamma radiation as 8 and 24 hours post exposure), showed a significant, time-dependent increase of HMGB1 (FIG. 5A), NLRP2 (FIG. 5B) and IL-1P (FIG. 5).

3.4. Thoracic Radiation of Mice Induces Time-Dependent Secretion of HMGB1 as Well as Induction of Gene Expression.

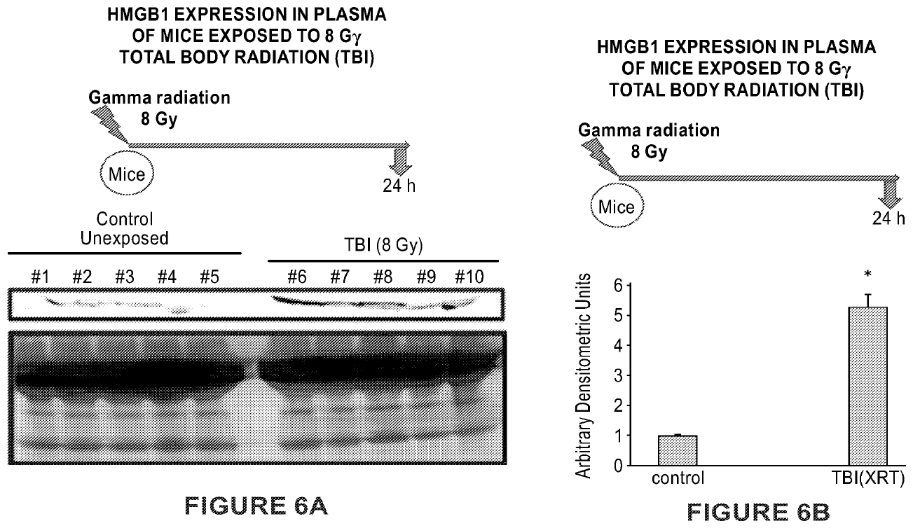

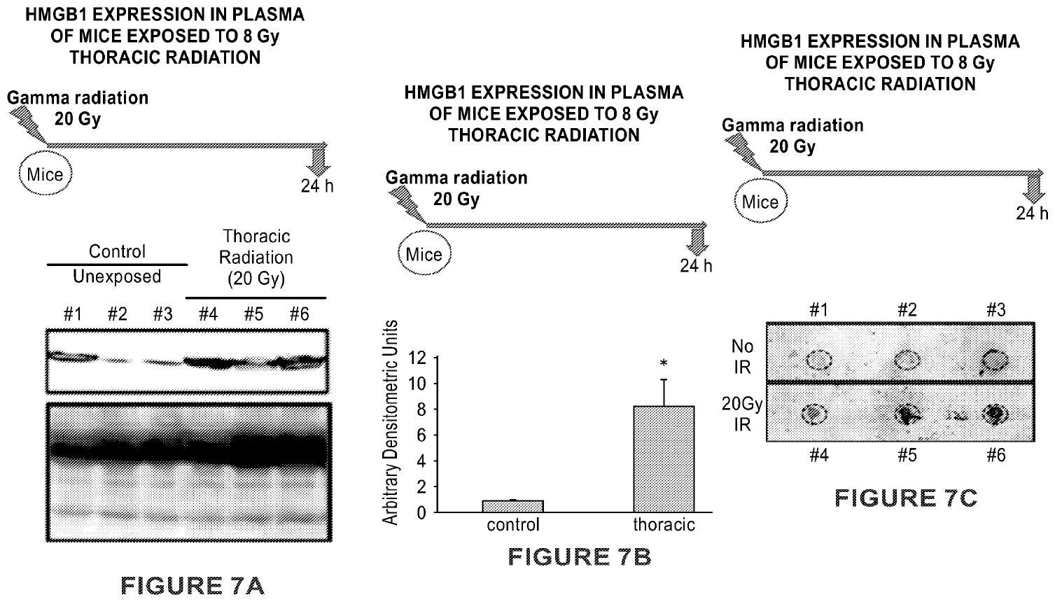

Plasma from mice (n=4 per cohort) exposed to 8 Gy (FIG. 6) and 20 Gy (n=3 per cohort) (FIG. 7) thoracic x-ray radiation were found to show a significant increase in HMGB1 levels, as seen in immunoblots and dot blots (FIG. 6 and FIG. 7, respectively).

3.5. Radiation Dose-Dependent Expression of IL-1β, NLRP3, IL-6 and sICAM Post Radiation Exposure in Mice.

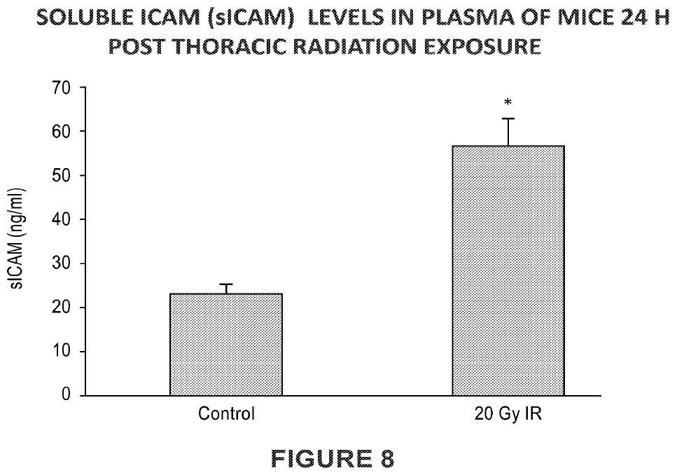

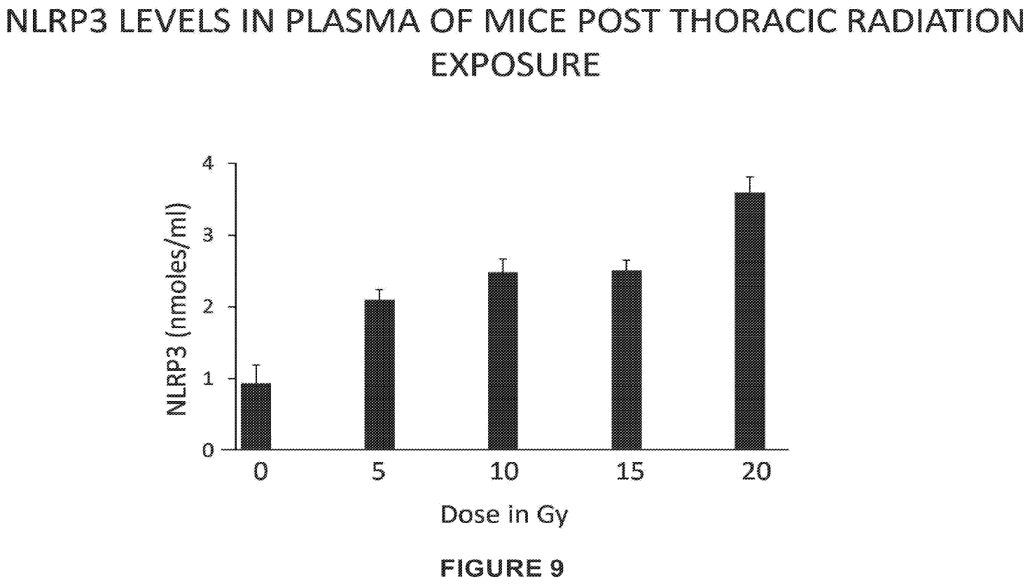

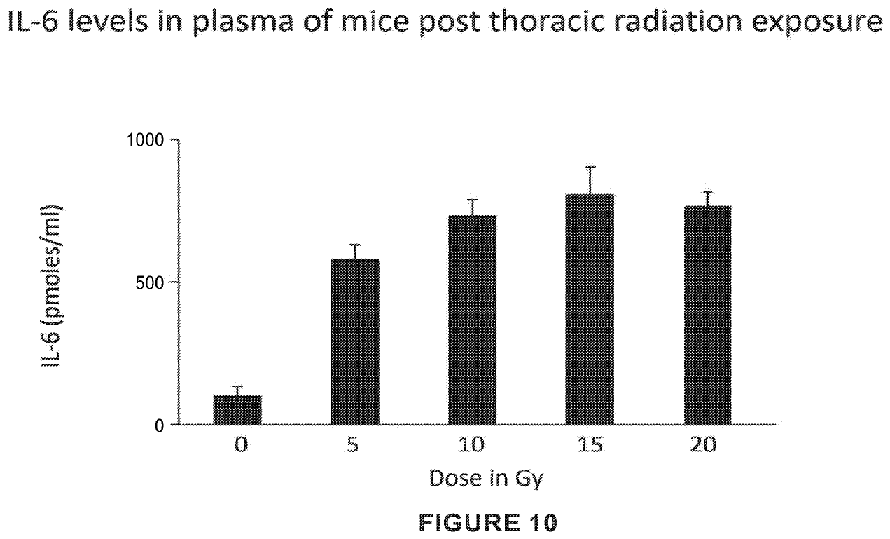

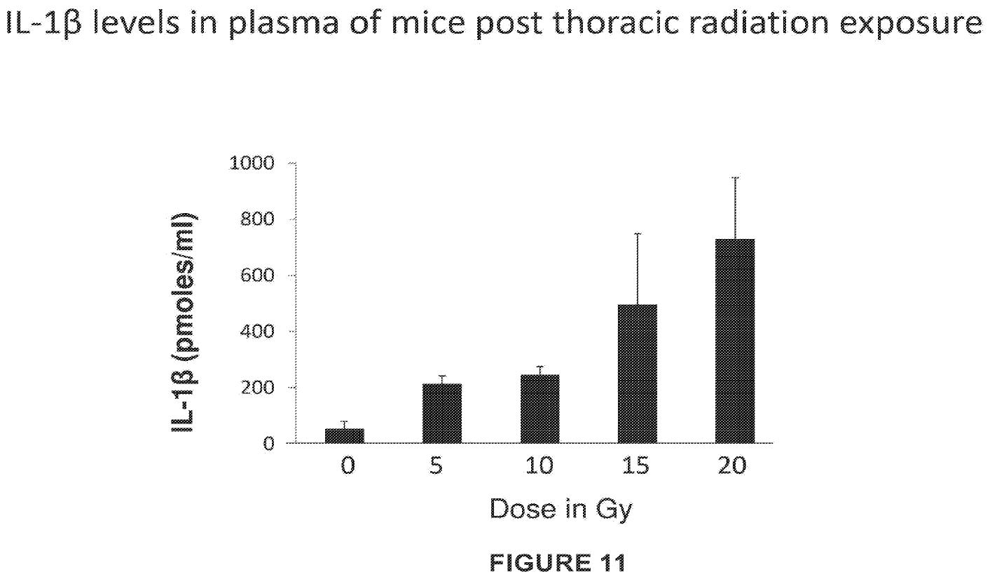

As observed in FIG. 8, we detected an increase in plasma levels of soluble ICAM-1 (sICAM), and NLRP3, IL-6, and IL-1β(FIGS. 9, 10, 11, respectively) in a dose-dependent manner.

3.6. Total Body Radiation (TBI) Exposure Induces a Radiation Dose-Dependent Increase in Expression of all 8 Named Biomarkers in Both Male and Female Mice.

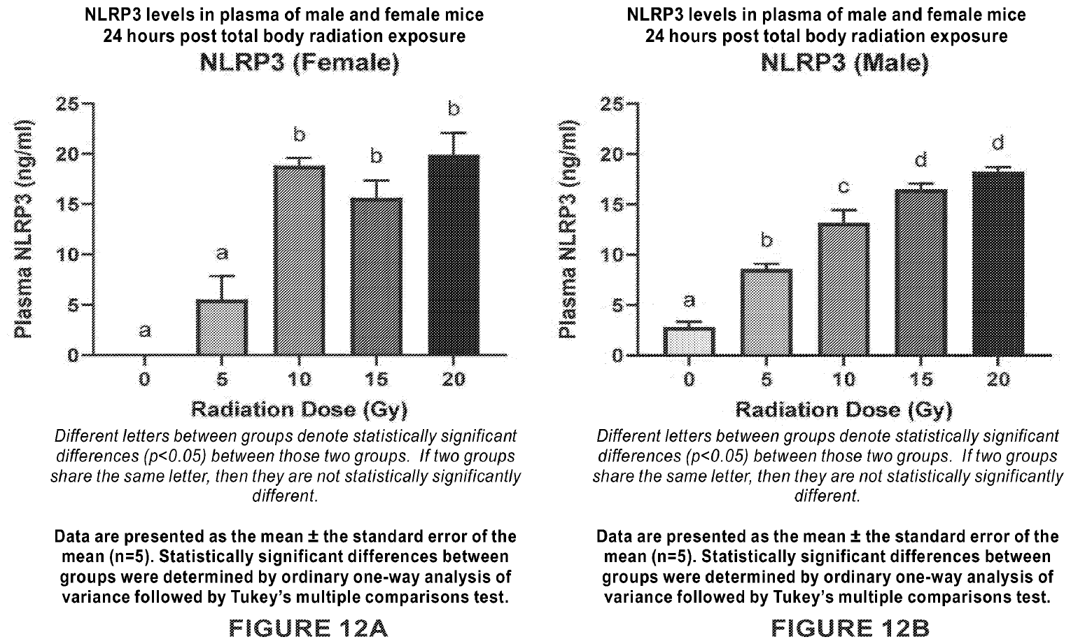

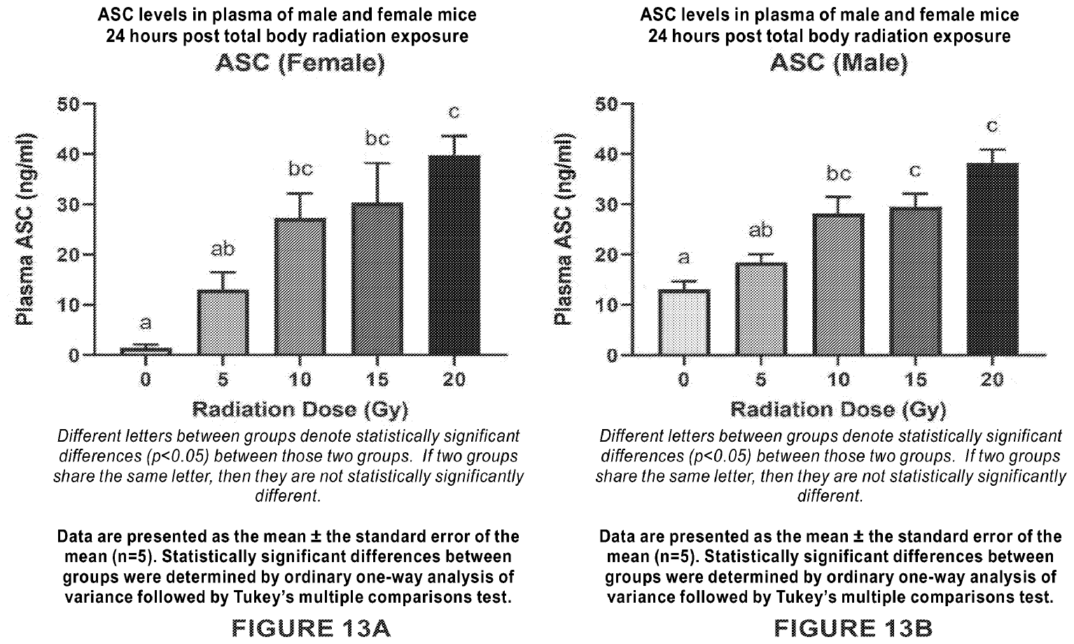

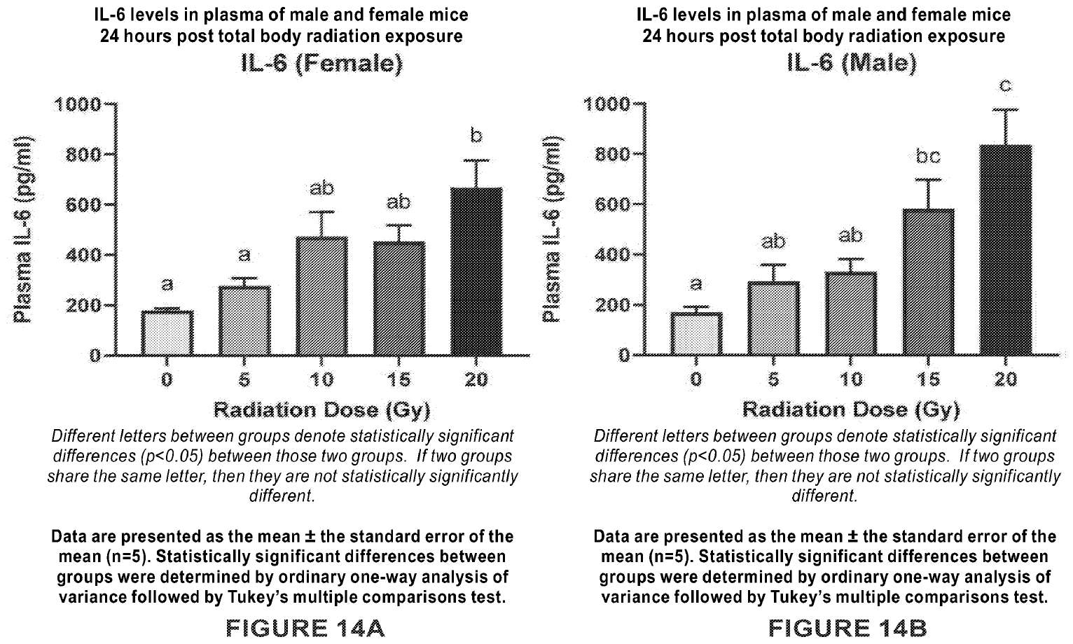

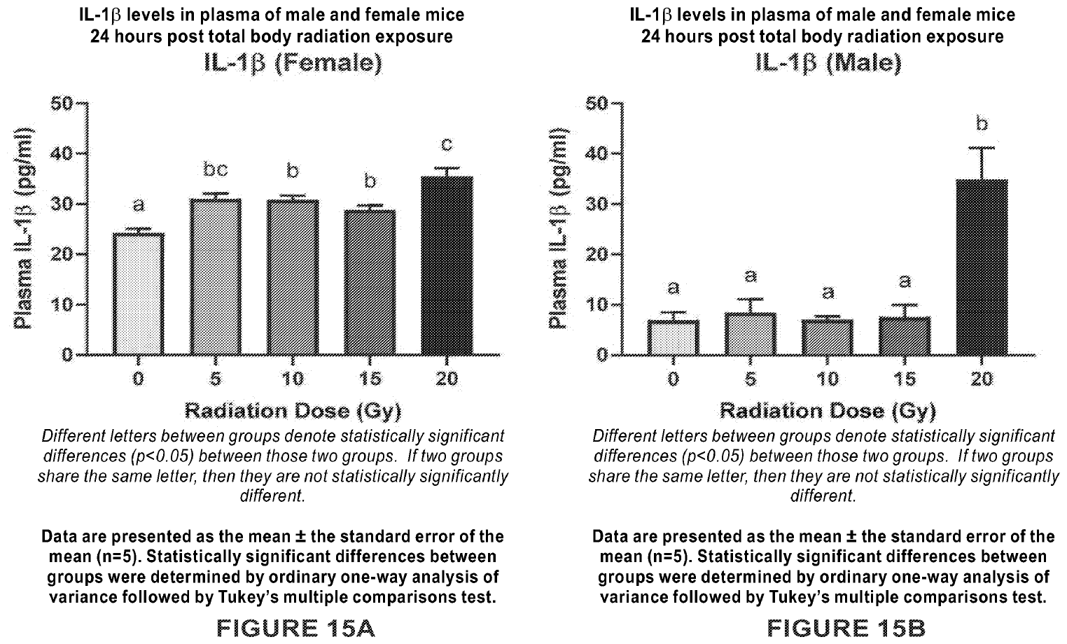

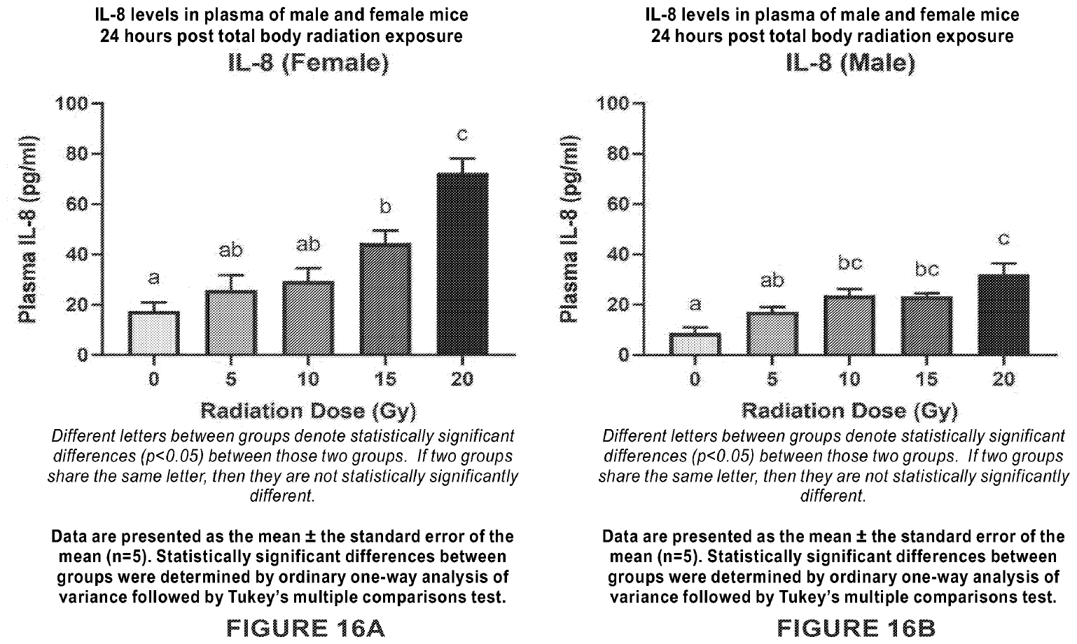

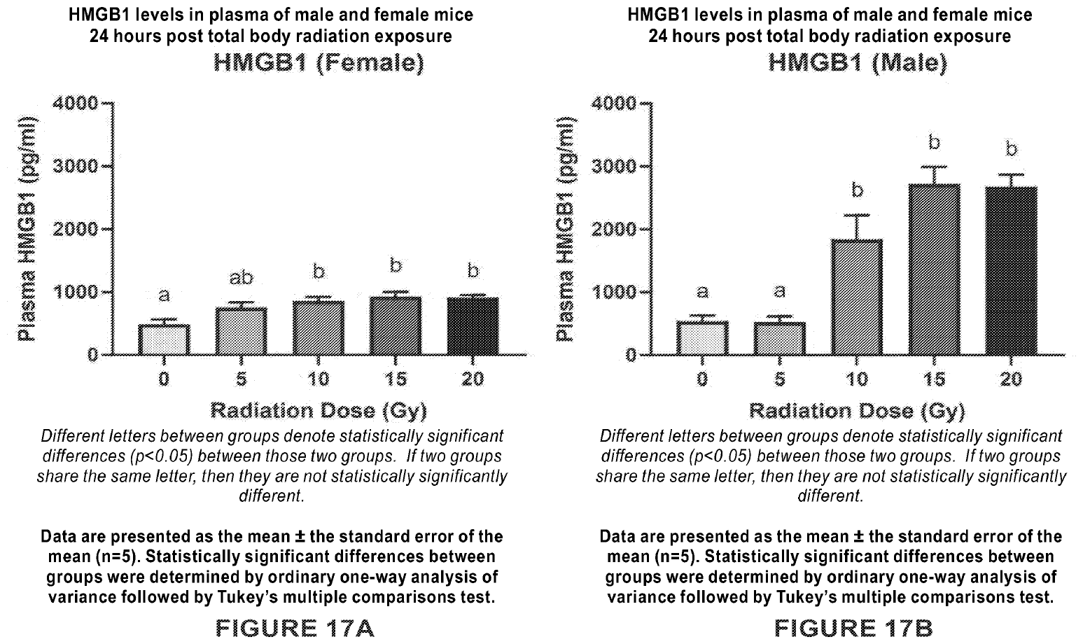

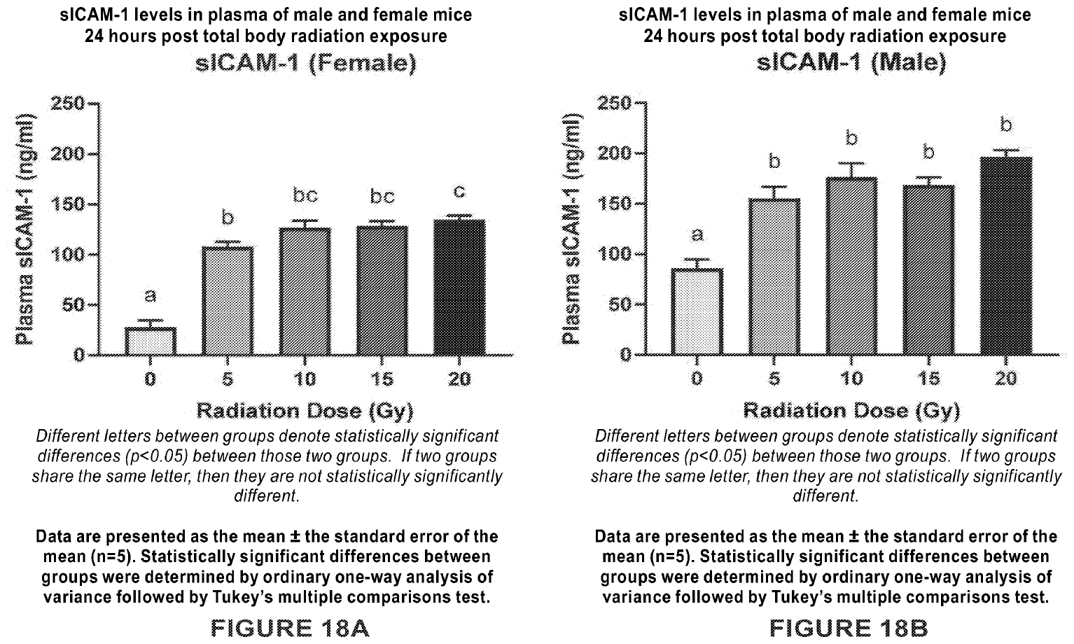

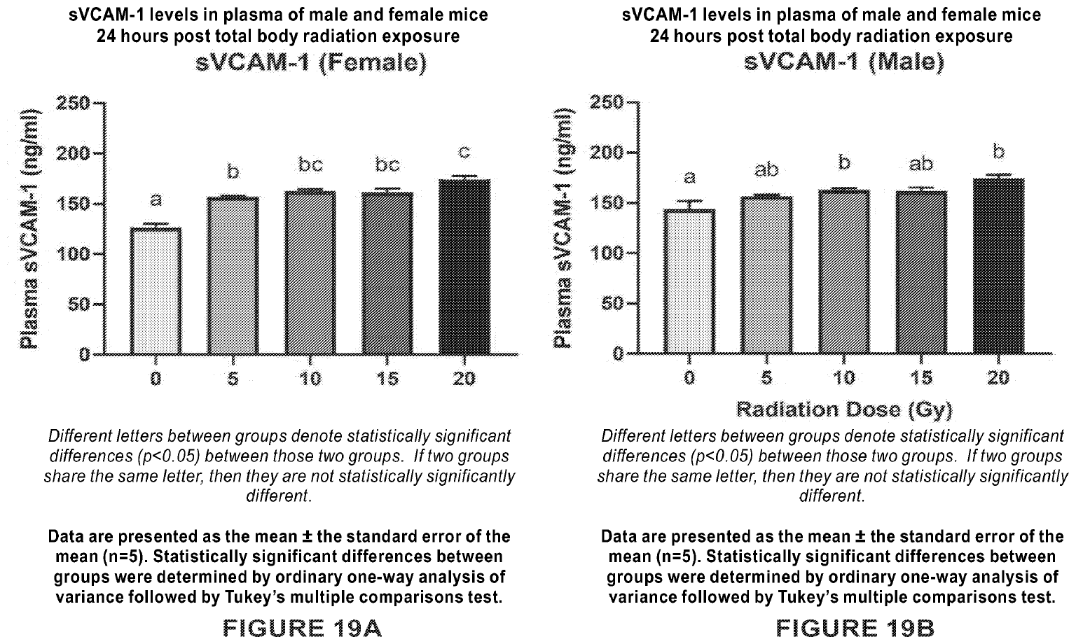

As observed in FIGS. 12-19, we detected an increase in plasma levels of NLRP3, ASC, I1-6, IL-1b, IL-8, HMGB1, sICAM-1 and sVCAM-1, respectively in a dose-dependent manner.

### CONCLUSION

We identified a panel of radiation-sensitive biomarkers that can be detected in biological fluids (cell supernatant and murine plasma) following exposure to increasing doses of radiation. These findings will form the basis of our eventual goal of a portable miniature platform that will allow rapid detection of inflammasome protein using a blood drop sample. Such a device for mass casualty screening whereby affected individuals (yes/no. i.e., irradiated/non-irradiated and level of exposure) can be detected in a single easy and rapid test (antibody-antigen reaction) would be an ideal biodosimeter.

### Example 2

Statistical Modeling for Predicting Radiation Exposure from a Panel of Blood Inflammasome Proteins

Further to the studies of Example 1 that confirmed a robust increase of all selected biomarkers in mouse plasma 24 hours post exposure to TBI, a statistical prediction model was developed with at least seven of the eight selected biodosimetry markers in the panel.

Specifically, one such biomarker is the NLRP3 inflammasome that has been reported to be activated upon radiation exposure. Activation of NLRP3 occurs via the damage associated molecular pattern (DAMP) protein HMGB1 (high mobility group box-1). Other biomarkers associated with the HMGB1-NLRP3 axis are adaptor protein ASC, interleukins IL-1, -6 and -8, soluble cellular adhesion molecules (CAMs) sICAM and sVCAM. When these biomarkers are highly expressed on the blood vessel wall, they eventually are “shed” into the circulation.

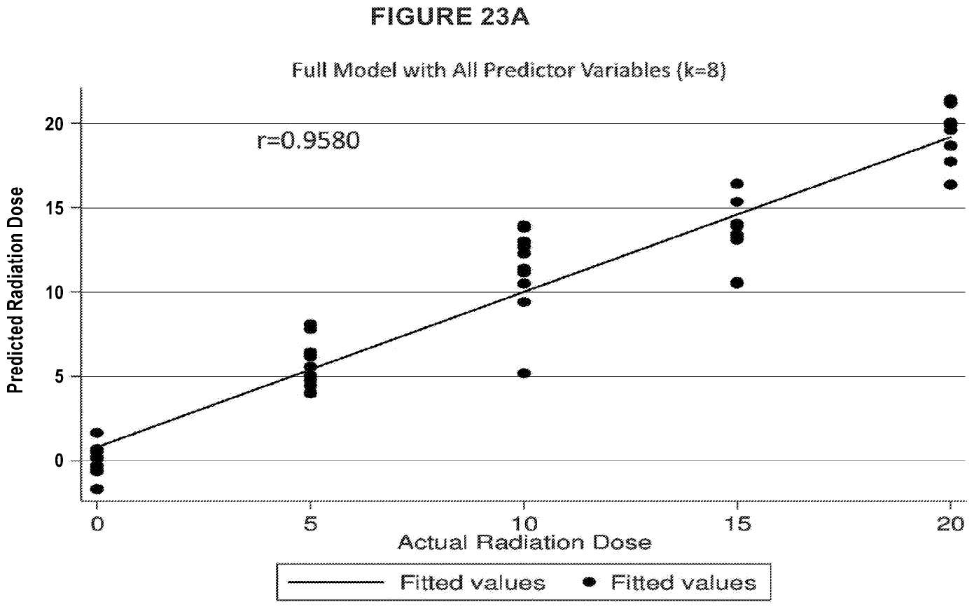

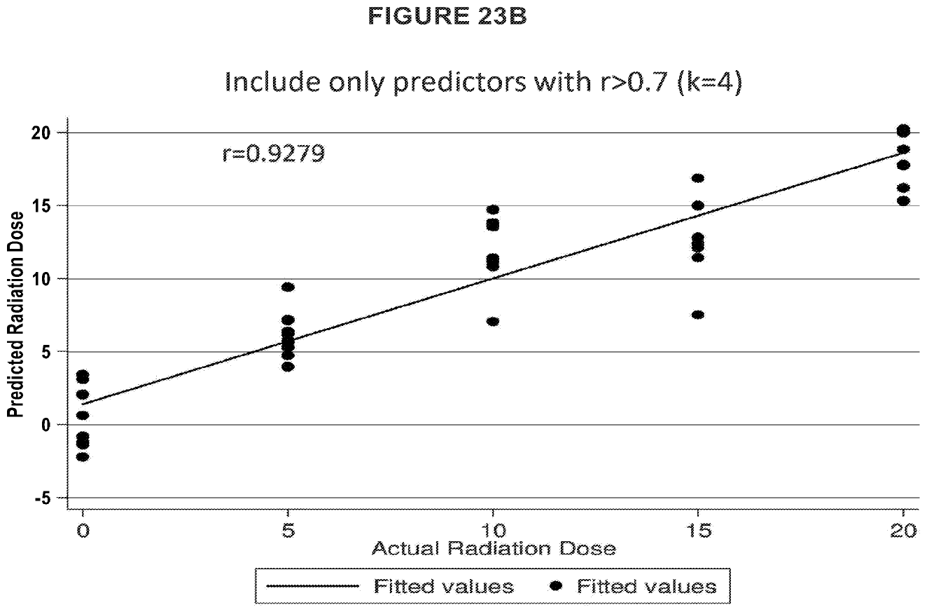

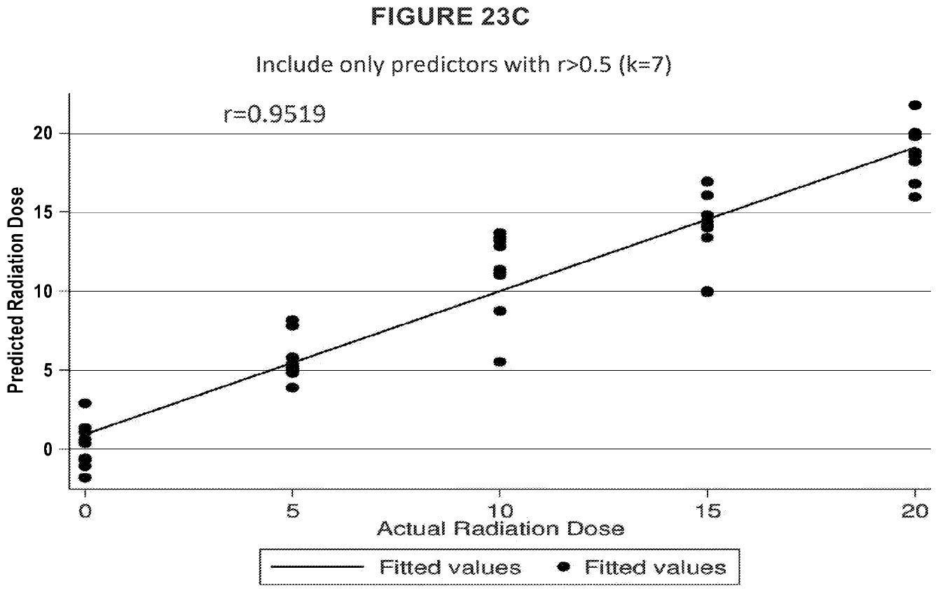

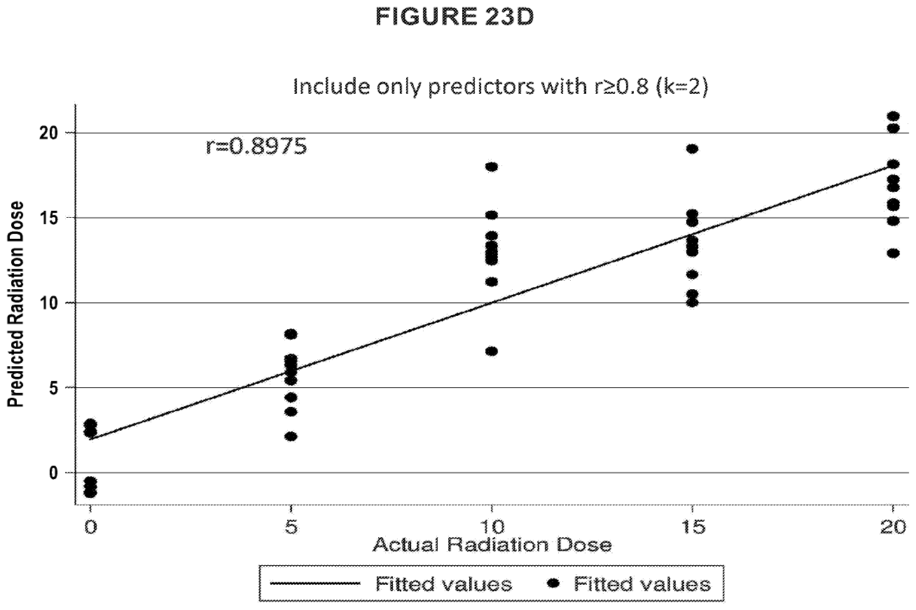

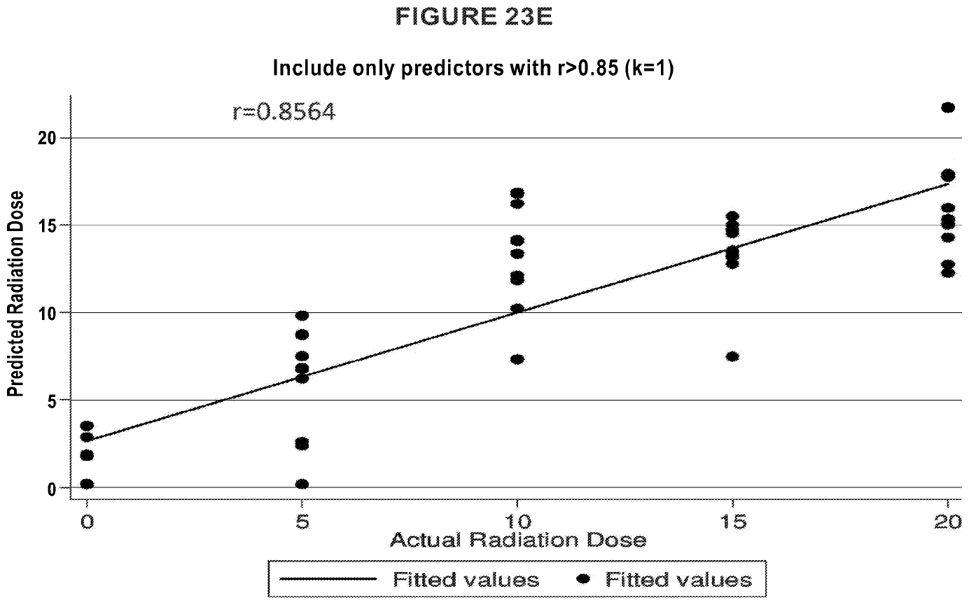

All 8 protein biomarkers were significantly upregulated at 24 h following radiation exposure and displayed a dose-dependent response to radiation, with NLRP3 (r=0.85, p<0.0001), ASC (r-0.80, p<0.0001), sVCAM-1 (r-0.77, p<0.0001), and IL-6 (r=0.74, p<0.0001) having the strongest correlation (r>0.70, p<0.05) with radiation dose. The multiple regression model for these data obtained through stepwise feature selection was predicted radiation dose=−13.810+0.187 (NLRP3)+0.109 (ASC)+0.005(IL-6)+0.083 (IL-8)+0.002 (HMGB1)+0.069 (sVCAM-1)+0.059 (IL-1). The correlation between the actual radiation dose and the predicted radiation dose for identified models is shown in FIGS. 23A-23E.

Based on mean absolute errors (MAE), Akaike information criterion with sample size correction (AICc), and Bayesian information criterion (BIC) scores, 4 models were generated that were highly predictive of radiation dose (r>0.9 for actual exposed dose vs. predicted dose). While the model containing all 8 biomarkers had the highest coefficient of determination (R2=0.92), the model generated from the stepwise feature selection process displayed the strongest information theoretic support. Overall, the quantitative, continuous dose prediction model determined by stepwise regression analysis explained >91% of the variation in radiation dose and displayed a Pearson correlation coefficient >0.95 for predicted versus actual radiation dose.

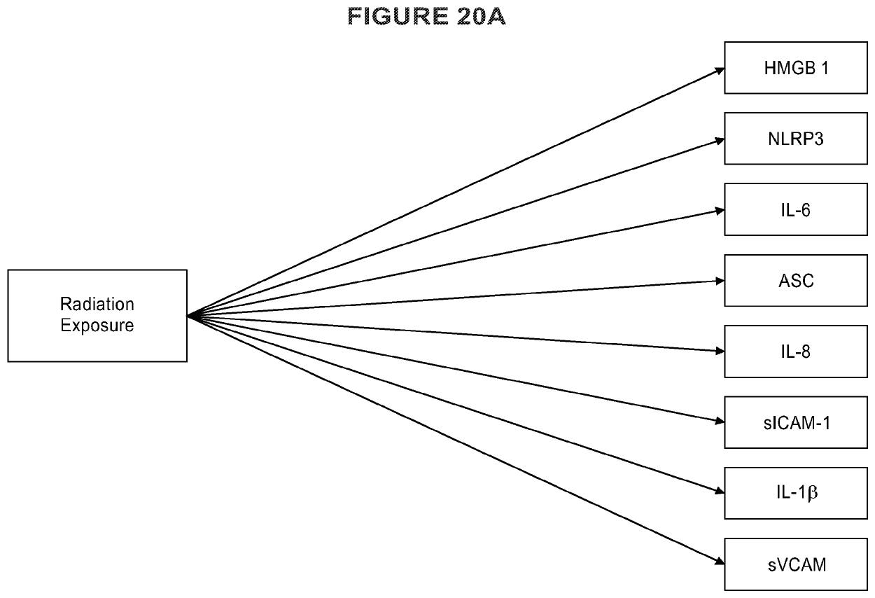

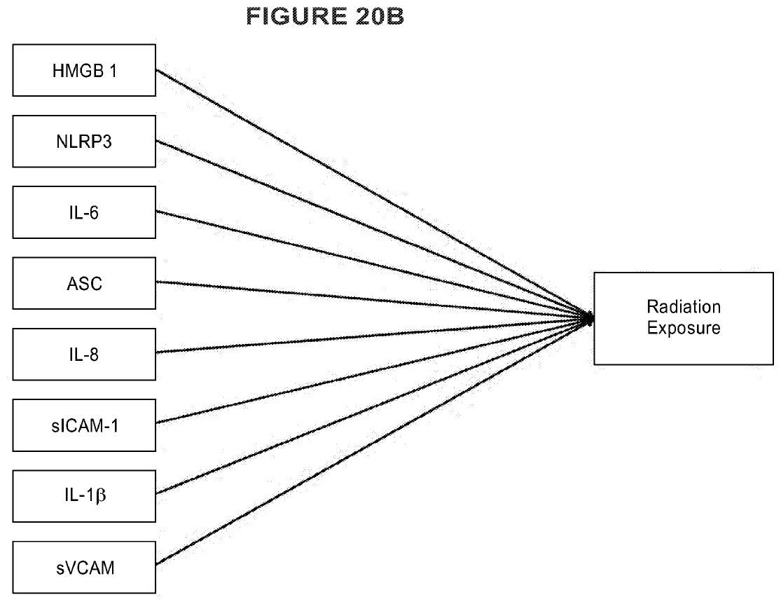

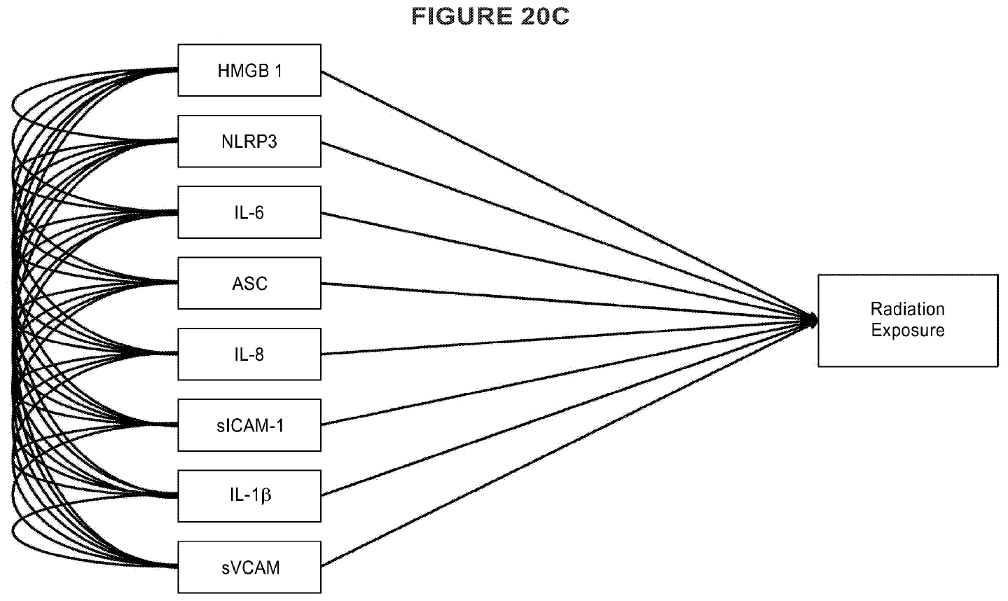

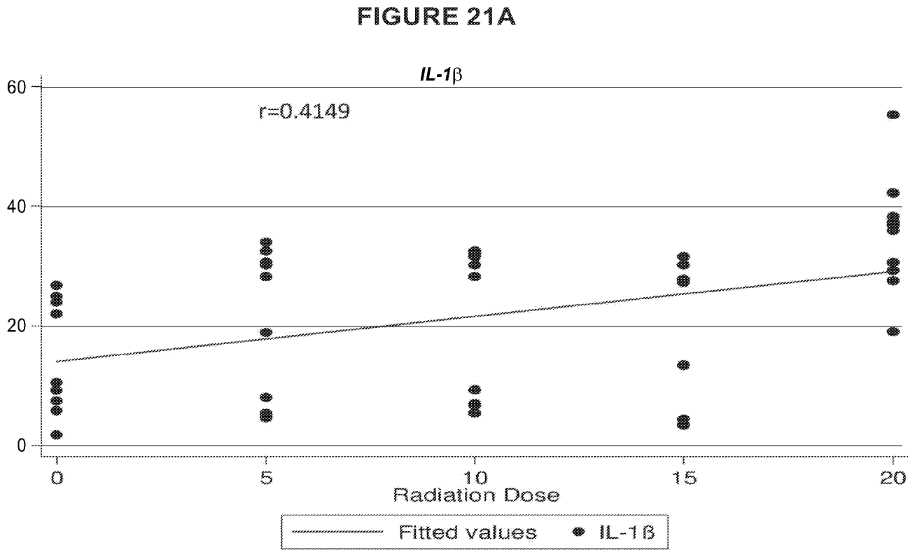

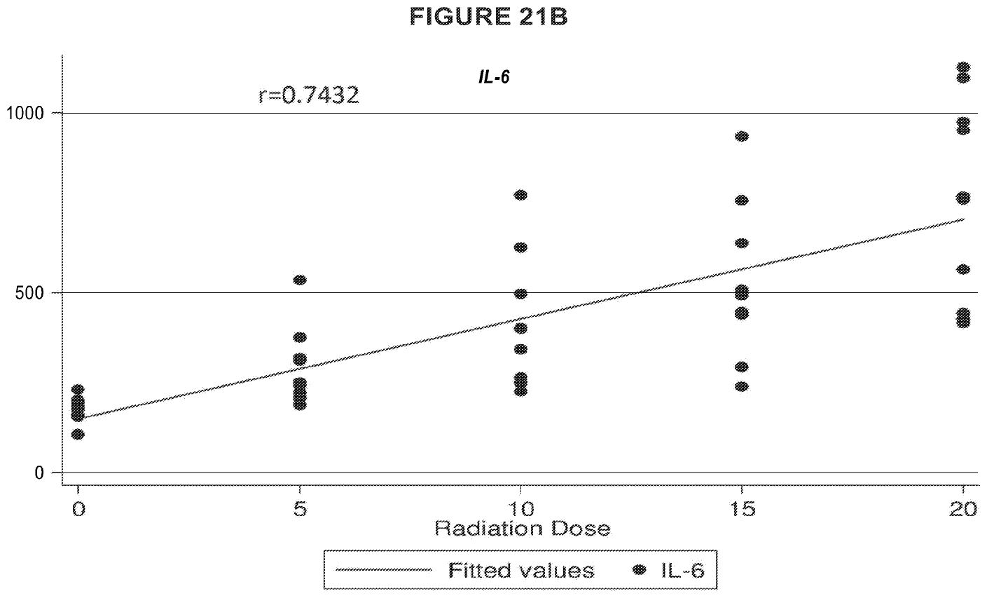

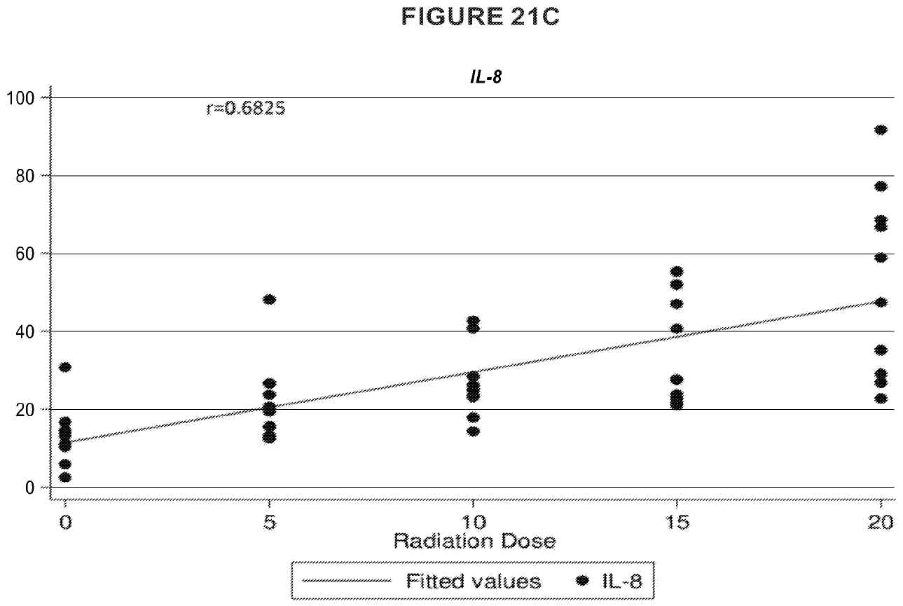

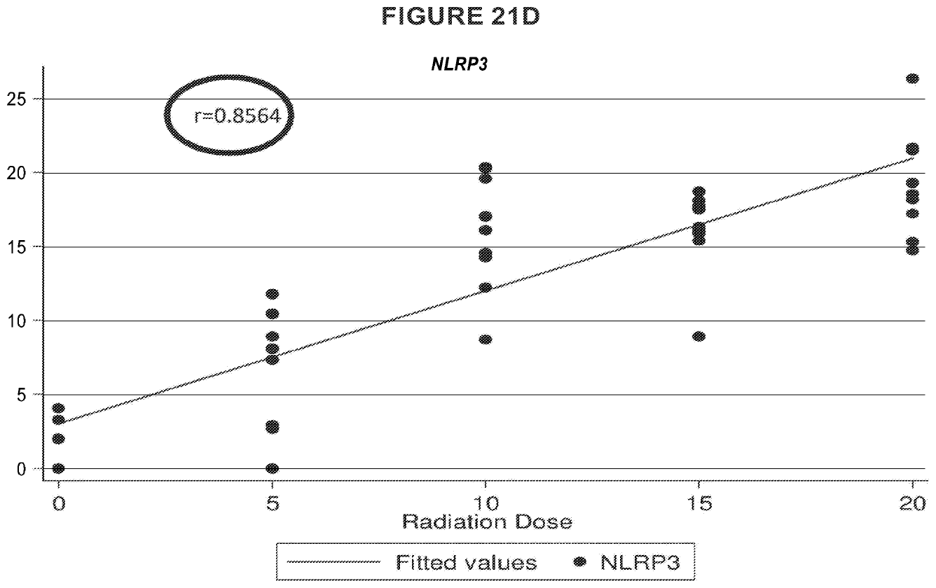

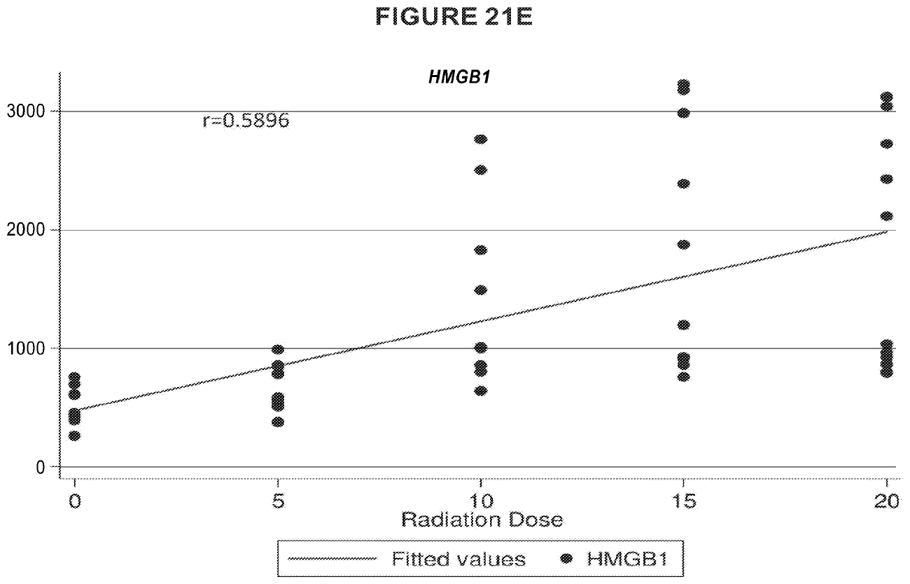

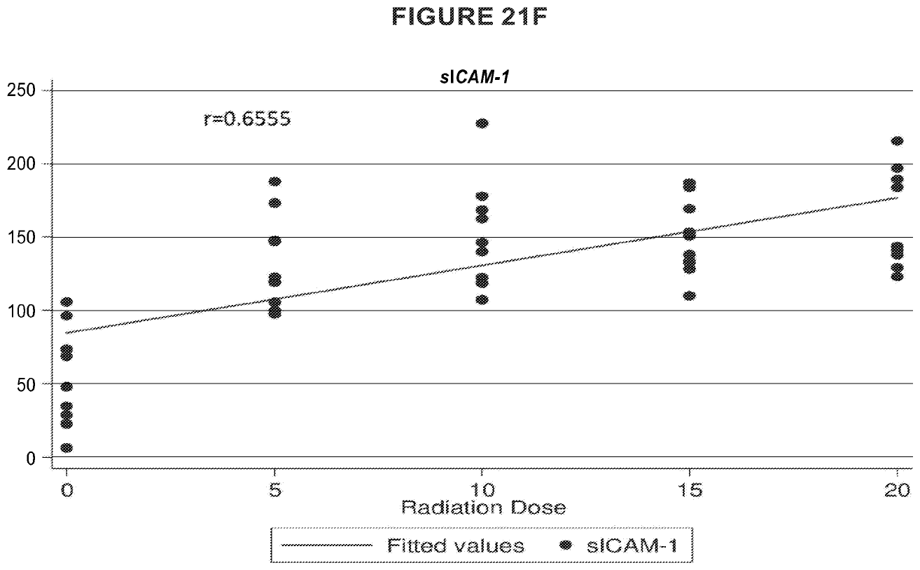

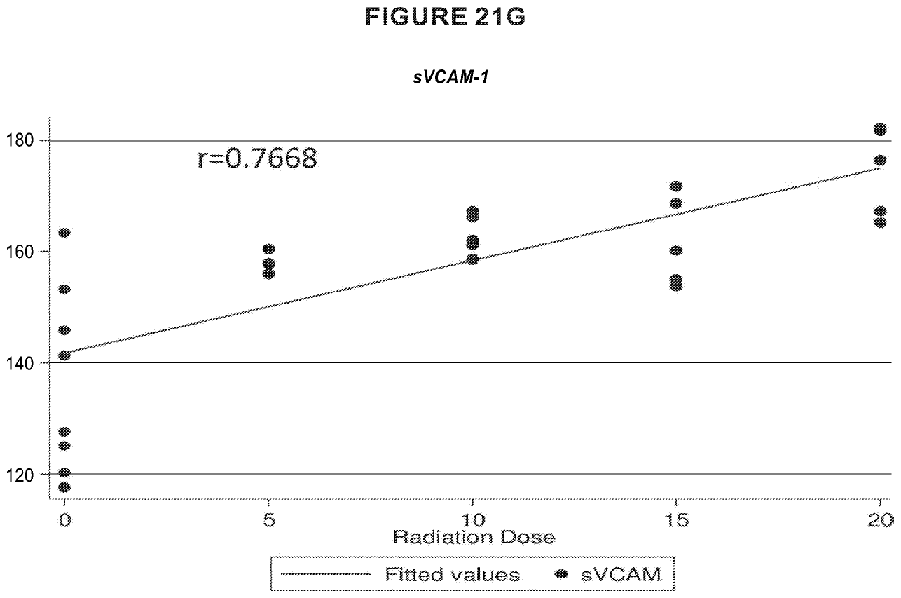

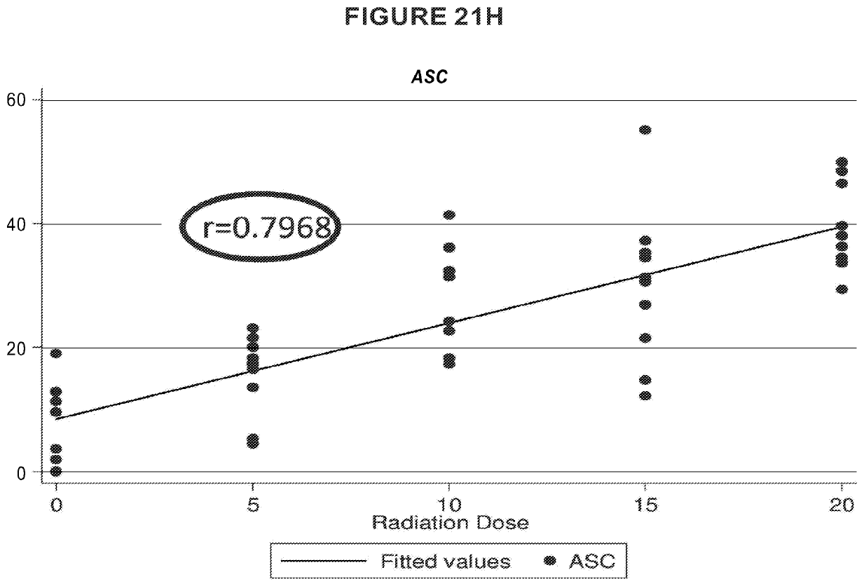

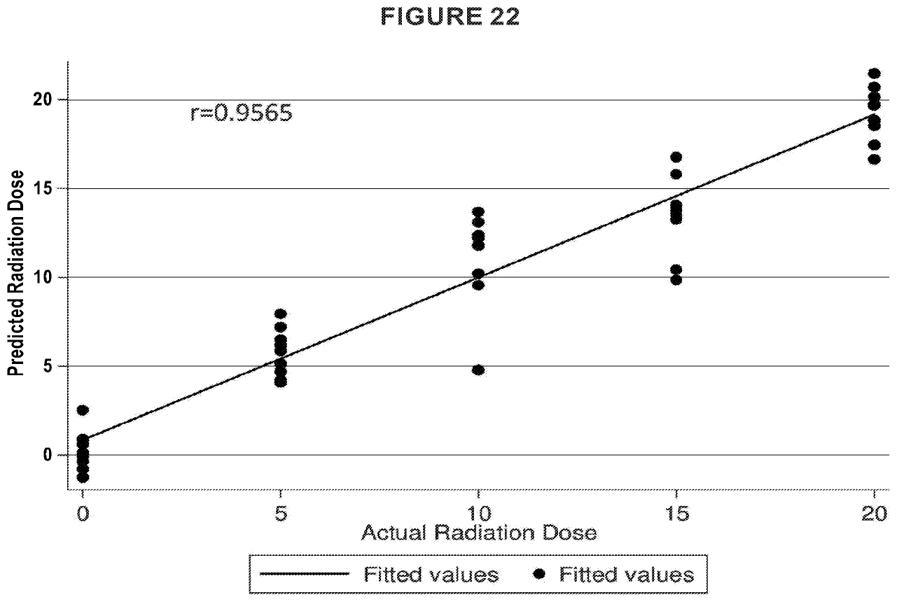

The present Example demonstrates that data-driven predictions can be made, through building a mathematical model, on radiation exposure using the blood levels of 8 inflammasome proteins, as shown in FIGS. 20A-20C and Tables 1-14.

TABLE 1
Biomarker Levels

| Biomarker | Unit | 0 Gy | 5 Gy | 10 Gy | 15 Gy | 20 Gy | p-value |
|---|---|---|---|---|---|---|---|
| IL-1β | (pg/ml) | 15.6 ± 9.5 | 19.8 ± 12.7 | 19.0 ± 12.7 | 18.3 ± 11.8 | 35.2 ± 9.7 | 0.003 |
| IL-6 | (pg/ml) | 175.6 ± 33.8 | 285.6 ± 105.8 | 402.3 ± 181.2 | 518.1 ± 208.8 | 752.3 ± 278.7 | <0.0001 |
| IL-8 | (pg/ml) | 13.2 ± 7.5 | 21.6 ± 10.4 | 26.7 ± 8.9 | 34.0 ± 13.4 | 52.4 ± 23.7 | <0.0001 |
| NLRP3 | (ng/ml) | 1.3 ± 1.6 | 7.1 ± 3.9 | 16.0 ± 3.7 | 16.1 ± 2.7 | 19.2 ± 3.6 | <0.0001 |
| HMGB1 | (pg/ml) | 514.3 ± 161.0 | 643.4 ± 211.9 | 1352.4 ± 771.7 | 1830.4 ± 1030.7 | 1800.3 ± 976.1 | 0.0002 |
| SICAM-1 | (ng/ml) | 53.8 ± 34.2 | 131.9 ± 31.0 | 151.7 ± 35.0 | 148.7 ± 25.1 | 162.3 ± 34.1 | <0.0001 |
| SVCAM-1 | (ng/ml) | 135.3 ± 16.0 | 157.2 ± 1.9 | 163.0 ± 3.4 | 161.8 ± 7.6 | 174.5 ± 7.5 | <0.0001 |
| ASC | (ng/ml) | 7.3 ± 6.6 | 15.8 ± 6.3 | 27.7 ± 8.9 | 29.9 ± 12.3 | 39.0 ± 7.0 | <0.0001 |

Data are presented as the mean±the standard deviation.

TABLE 2A
Pearson's Correlation Coefficients

| | Correlation | |
|---|---|---|
| Biomarker | Coefficient (r) | p-value |
| IL-1β | 0.4149 | 0.0027 |
| IL-6 | 0.7432 | <0.0001 |
| IL-8 | 0.6825 | <0.0001 |
| NLRP3 | 0.8564 | <0.0001 |
| HMGB1 | 0.5896 | <0.0001 |
| sICAM-1 | 0.6555 | <0.0001 |
| sVCAM-1 | 0.7668 | <0.0001 |
| ASC | 0.7968 | <0.0001 |

All 8 blood biomarkers have a high dose-dependent response to radiation exposure

TABLE 2B
Pearson's Correlation Coefficients - Coefficient
Value and Strength of Association

| | Strength of |
|---|---|
| Coefficient Value | Association * Cohen (1988) |
| 0.1 < r < 0.3 | small correlation |
| 0.3 < r < 0.5 | medium/moderate correlation |
| r ≥ 0.5 | large/strong correlation |

TABLE 2C
Variable Selection Method
Univariate analysis
Any variable with a significant univariate test is selected as a
candidate for multivariate analysis.
Multivariate analysis
Statistically control for the effect of other predictor variables
Observe the effect of one predictor variable by holding the other
predictor variables constant.
Stepwise selection method
Effects are entered into and removed from the model

TABLE 3
Simple Linear Regression Analysis Results

| Biomarker | Unit | Coefficient | SE | p-value | R² |
|---|---|---|---|---|---|
| IL-1β | (pg/ml) | 0.2288 | 0.0724 | 0.0030 | 0.1722 |
| IL-6 | (pg/ml) | 0.0199 | 0.0026 | <0.0001 | 0.5523 |
| IL-8 | (pg/ml) | 0.2566 | 0.0397 | <0.0001 | 0.4659 |
| NLRP3 | (ng/ml) | 0.8170 | 0.0726 | <0.0001 | 0.7334 |
| HMGB1 | (pg/ml) | 0.0046 | 0.0009 | <0.0001 | 0.3476 |
| sICAM-1 | (ng/ml) | 0.0932 | 0.0158 | <0.0001 | 0.4296 |
| sVCAM-1 | (ng/ml) | 0.3540 | 0.0428 | <0.0001 | 0.5880 |
| ASC | (ng/ml) | 0.4093 | 0.0448 | <0.0001 | 0.6350 |

R² value represents the % of the variance in radiation exposure accounted for by the predictor variable.

The regression coefficient states the expected increase in radiation dose for every one (1) unit increase in the predictor variable.

| | | | | | Beta | |
|---|---|---|---|---|---|---|
| | | Coef- | | | coef- | |
| Biomarker | Unit | ficient | SE | p-value | ficient | R² |
| NLRP3 | (ng/ml) | 0.1872 | 0.0848 | 0.033 | 0.1962 | 0.9149 |
| ASC | (ng/ml) | 0.1090 | 0.0365 | 0.005 | 0.2191 | |
| IL-6 | (pg/ml) | 0.0047 | 0.0018 | 0.010 | 0.1753 | |
| IL-8 | (pg/ml) | 0.0835 | 0.0259 | 0.002 | 0.2245 | |
| HMGB1 | (pg/ml) | 0.0020 | 0.0005 | <0.001 | 0.2462 | |
| SVCAM-1 | (ng/ml) | 0.0685 | 0.0316 | 0.036 | 0.1501 | |
| IL-1β | (pg/ml) | 0.0589 | 0.0290 | 0.049 | 0.1100 | |
| SICAM-1 | (ng/ml) | — | — | — | — | |

All variables in the model are predictive independently of any of the other variables in the analysis (predictive ability is not due to its association with other predictors).
91.49% of the variance in radiation dose is accounted for by the 7 biomarker variables in the model.

TABLE 5
Information Criterion

| | # of Variables | | | | | |
|---|---|---|---|---|---|---|
| Model Description | in Model | coefficient | R² (p-value) | MAE | AICc | BIC |
| Stepwise Regression | 7 | 0.9565 | 0.9149 (<0.0001) | 1.5527 | 224.4401 | 234.673 |
| Analysis | | | | | | |

MAE: mean absolute error
AICc: corrected Akaike information criterion
AIC: Akaike information criterion
BIC: Bayesian information criterion

TABLE 6
Radiation Dose Prediction at 0 Gy

| Sample | | Actual Dose | Predicted Dose | |
|---|---|---|---|---|
| # | Sex | (Gy) | (Gy) | Difference |
| 1 | Male | 0 | — | — |
| 2 | Male | 0 | 0.67 | −0.67 |
| 3 | Male | 0 | 0.89 | −0.89 |
| 4 | Male | 0 | −0.37 | 0.37 |
| 5 | Male | 0 | 2.54 | −2.54 |
| | | Average | 0.93 | −0.93 |
| | | SD | 1.2 | 1.2 |
| 1 | Female | 0 | −0.77 | 0.77 |
| 2 | Female | 0 | 0.14 | −0.14 |
| 3 | Female | 0 | −0.1 | 0.1 |
| 4 | Female | 0 | −1.23 | 1.23 |
| 5 | Female | 0 | 0.63 | −0.63 |
| | | Average | −0.26 | 0.26 |
| | | SD | 0.74 | 0.74 |

TABLE 7
Radiation Dose Prediction at 5 Gy

| Sample | | Actual Dose | Predicted Dose | |
|---|---|---|---|---|
| # | Sex | (Gy) | (Gy) | Difference |
| 6 | Male | 5 | 4.17 | 0.83 |
| 7 | Male | 5 | 5.18 | −0.18 |
| 8 | Male | 5 | 4.08 | 0.92 |
| 9 | Male | 5 | 4.21 | 0.79 |
| 10 | Male | 5 | 7.19 | −2.19 |
| | | Average | 4.96 | 0.04 |
| | | SD | 1.32 | 1.32 |
| 6 | Female | 5 | 7.94 | −2.94 |
| 7 | Female | 5 | 6.17 | −1.17 |
| 8 | Female | 5 | 4.7 | 0.3 |
| 9 | Female | 5 | 5.83 | −0.83 |
| 10 | Female | 5 | 6.49 | −1.49 |
| | | Average | 6.23 | −1.23 |
| | | SD | 1.17 | 1.17 |

TABLE 8
Radiation Dose Prediction at 10 Gy

| Sample | | Actual Dose | Predicted Dose | |
|---|---|---|---|---|
| # | Sex | (Gy) | (Gy) | Difference |
| 11 | Male | 10 | 4.79 | 5.21 |
| 12 | Male | 10 | 10.19 | −0.19 |
| 13 | Male | 10 | 12.2 | −2.2 |
| 14 | Male | 10 | 13.1 | −3.1 |
| 15 | Male | 10 | 12.36 | −2.36 |
| | | Average | 10.53 | −0.53 |
| | | SD | 3.38 | 3.38 |
| 11 | Female | 10 | 13.68 | −3.68 |
| 12 | Female | 10 | 13.67 | −3.67 |
| 13 | Female | 10 | 9.54 | 0.46 |
| 14 | Female | 10 | 11.81 | −1.81 |
| 15 | Female | 10 | 11.76 | −1.76 |
| | | Average | 12.09 | −2.09 |
| | | SD | 1.71 | 1.71 |

TABLE 9
Radiation Dose Prediction at 15 Gy

| Sample | | Actual Dose | Predicted Dose | |
|---|---|---|---|---|
| # | Sex | (Gy) | (Gy) | Difference |
| 16 | Male | 15 | 13.26 | 1.74 |
| 17 | Male | 15 | 13.77 | 1.23 |
| 18 | Male | 15 | 13.97 | 1.03 |
| 19 | Male | 15 | 14.03 | 0.97 |
| 20 | Male | 15 | 15.79 | −0.79 |
| | | Average | 14.16 | 0.84 |
| | | SD | 0.96 | 0.96 |
| 16 | Female | 15 | 13.5 | 1.5 |
| 17 | Female | 15 | 9.85 | 5.15 |
| 18 | Female | 15 | 14.07 | 0.93 |
| 19 | Female | 15 | 10.45 | 4.55 |
| 20 | Female | 15 | 16.76 | −1.76 |
| | | Average | 12.93 | 2.07 |
| | | SD | 2.83 | 2.83 |

TABLE 10
Radiation Dose Prediction at 20 Gy

| Sample | | Actual Dose | Predicted Dose | |
|---|---|---|---|---|
| # | Sex | (Gy) | (Gy) | Difference |
| 21 | Male | 20 | 20.7 | −0.7 |
| 22 | Male | 20 | 18.53 | 1.47 |
| 23 | Male | 20 | 19.71 | 0.29 |
| 24 | Male | 20 | — | — |
| 25 | Male | 20 | 17.45 | 2.55 |
| | | Average | 19.1 | 0.9 |
| | | SD | 1.41 | 1.41 |
| 21 | Female | 20 | 20.12 | −0.12 |
| 22 | Female | 20 | 18.83 | 1.17 |
| 23 | Female | 20 | 16.64 | 3.36 |
| 24 | Female | 20 | 19.65 | 0.35 |
| 25 | Female | 20 | 21.46 | −1.46 |
| | | Average | 19.34 | 0.66 |
| | | SD | 1.78 | 1.78 |

TABLE 11
Radiation Dose Prediction

| | Average | | |
|---|---|---|---|
| Actual Dose | Predicted Dose | Standard | |
| (Gy) | (Gy) | Deviation | p-value |
| 0 | 0.27 | 1.1 | 0.486 |
| 5 | 5.6 | 1.35 | 0.1975 |
| 10 | 11.31 | 2.66 | 0.1536 |
| 15 | 13.55 | 2.09 | 0.0555 |
| 20 | 19.23 | 1.53 | 0.1715 |

No statistically significant difference between the actual dose and predicted dose.

| | # of Variables | | | | | |
|---|---|---|---|---|---|---|
| Model Description | in Model | coefficient | R² (p-value) | MAE | AICc | BIC |
| Stepwise Regression | 7 | 0.9565 | 0.9149 (<0.0001) | 1.5527 | 224.4401 | 234.673 |
| Analysis | | | | | | |
| Full Model with | 8 | 0.9580 | 0.9177 (<0.0001) | 1.5305 | 226.006 | 236.9009 |
| All Predictors | | | | | | |
| Include only predictors | 4 | 0.9279 | 0.8611 (<0.0001) | 2.0838 | 239.2659 | 246.5732 |
| with r > 0.7 | | | | | | |
| Include only predictors | 7 | 0.9519 | 0.9062 (<0.0001) | 1.6058 | 229.1081 | 239.3409 |
| with r > 0.5 | | | | | | |
| Include only predictors | 2 | 0.8975 | 0.8054 (<0.0001) | 2.5316 | 250.3091 | 254.9925 |
| with r ≥ 0.8 | | | | | | |
| Include only predictors | 1 | 0.8564 | 0.7334 (<0.0001) | 2.8486 | 263.0428 | 266.2397 |
| with r ≥ 0.85 | | | | | | |

TABLE 13
Dose Prediction Table - Model Comparison

| | # of Biomarkers | | | | | |
|---|---|---|---|---|---|---|
| Model Description | in the Model | 0 Gy | 5 Gy | 10 Gy | 15 Gy | 20 Gy |
| Stepwise Regression | 7 | 0.27 ± 1.10 | 5.60 ± 1.35 | 11.31 ± 2.66 | 13.55 ± 2.09 | 19.23 ± 1.53 |
| Analysis | | | | | | |
| Full Model with | 8 | 0.25 ± 0.94 | 5.86 ± 1.36 | 11.33 ± 2.59 | 13.44 ± 1.83 | 19.27 ± 1.61 |
| All Predictors | | | | | | |
| Include only predictors | 4 | 0.14 ± 2.23 | 6.15 ± 1.52 | 12.10 ± 2.27 | 13.23 ± 2.82 | 18.21 ± 1.73 |
| with r > 0.7 | | | | | | |
| Include only predictors | 7 | 0.23 ± 1.44 | 5.62 ± 1.33 | 11.42 ± 2.59 | 13.77 ± 2.27 | 18.86 ± 1.78 |
| with r > 0.5 | | | | | | |
| Include only predictors | 2 | 0.65 ± 1.87 | 5.73 ± 1.92 | 12.98 ± 2.76 | 13.44 ± 2.58 | 16.95 ± 2.56 |
| with r ≥ 0.8 | | | | | | |
| Include only predictors | 1 | 1.23 ± 1.32 | 5.97 ± 3.18 | 13.28 ± 3.04 | 13.34 ± 2.24 | 15.88 ± 2.93 |
| with r ≥ 0.85 | | | | | | |

TABLE 14
Information Criterion of Additional Alternative Models

| | | R² | | | |
|---|---|---|---|---|---|
| Model Description | coefficient | (p-value) | MAE | AICc | BIC |
| Dose~NLRP3 + ASC + | 0.9565 | 0.9149 | 1.5500 | 224.4400 | 234.6700 |
| IL-6 + IL-8 + HMGB1 + | | (<0.0001) | | | |
| sVCAM-1 + IL-1β | | | | | |
| Dose~NLRP3 + ASC | 0.8975 | 0.8054 | 2.5316 | 250.3091 | 254.9925 |
| | | (<0.0001) | | | |
| Dose~NLRP3 + ASC + IL-6 | 0.9205 | 0.8473 | 1.6690 | 241.1812 | 247.2374 |
| | | (<0.0001) | | | |
| Dose~NLRP3 + ASC + IL-8 | 0.9126 | 0.8328 | 2.3888 | 245.5258 | 251.5820 |
| | | (<0.0001) | | | |
| Dose~NLRP3 + ASC + IL-6 + | 0.9301 | 0.8651 | 1.9711 | 237.8401 | 245.1473 |
| IL-8 | | (<0.0001) | | | |
| Dose~ASC | 0.7968 | 0.6350 | 3.4617 | 291.6286 | 294.9309 |
| | | (<0.0001) | | | |
| Dose~NLRP3 | 0.8564 | 0.7334 | 2.8486 | 263.0428 | 266.2397 |
| | | (<0.0001) | | | |
| Dose~ASC + IL-6 | 0.8926 | 0.7967 | 2.4921 | 264.7232 | 269.5703 |
| | | (<0.0001) | | | |
| Dose~ASC + IL-8 | 0.8547 | 0.7306 | 3.0316 | 278.8121 | 283.6592 |
| | | (<0.0001) | | | |
| Dose~NLRP3 + IL-6 | 0.8801 | 0.7746 | 2.6146 | 257.3780 | 262.0614 |
| | | (<0.0001) | | | |
| Dose~NLRP3 + IL-8 | 0.8832 | 0.7801 | 2.5839 | 256.1873 | 260.8707 |
| | | (<0.0001) | | | |
| Dose~NLRP3 + IL-6 + | 0.8995 | 0.8091 | 2.3036 | 251.8865 | 257.9427 |
| IL-8 | | (<0.0001) | | | |
| Dose~ASC + IL-6 + | 0.9121 | 0.8320 | 2.2234 | 257.6776 | 263.9621 |
| IL-8 | | (<0.0001) | | | |
| Dose~IL-6 | 0.7432 | 0.5523 | 3.9745 | 301.8313 | 305.1336 |
| | | (<0.0001) | | | |
| Dose~IL-6 + IL-8 | 0.8247 | 0.6802 | 3.2378 | 287.3806 | 292.2277 |
| | | (<0.0001) | | | |
| Dose~IL-8 | 0.6825 | 0.4659 | 4.1725 | 310.6610 | 313.9633 |
| | | (<0.0001) | | | |

All patents, patent applications, and scientific publications cited herein are hereby incorporated by reference in their entirety.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of detecting whether a subject has been exposed to ionizing radiation, comprising:
a) obtaining a biological sample from the subject; and
b) measuring levels of one or more inflammasome biomarkers selected from the group consisting of NLRP3, ASC, HMGB1, IL-1β, IL-6, IL-8, sICAM, and sVCAM in the biological sample, wherein a change in levels as compared to a non-irradiated control indicates the subject has been exposed to ionizing radiation.

2. The method of claim 1, wherein the level is a protein level.

3. The method of claim 1, wherein the level is an mRNA level.

4. The method of claim 1, wherein measuring comprises ELISA, immunoassays, immunostaining and fluorescence assays, western blot, or dot blot, immunoprecipitation, RT-PCR, nucleic acid hybridization, or a combination thereof.

5. The method of claim 1, wherein elevated levels of HMGB1 and/or sICAM-1 as compared to the control indicates the subject has been exposed to ionizing radiation.

6. The method of claim 1, wherein elevated levels of three or more of the inflammasome biomarkers as compared to the control indicates the subject has been exposed to ionizing radiation.

7. The method of claim 1, wherein the measuring occurs 8 hours after exposure.

8. The method of claim 1, wherein the measuring occurs 24 hours after exposure.

9. The method of claim 1, wherein the measuring occurs 1 to 14 days after exposure.

10. The method of claim 1, wherein the biological sample comprises blood.

11. The method of claim 1, further comprising treating the subject for radiation exposure.

12. The method of claim 1, wherein the subject is a human.

13. The method of claim 1, wherein the subject has been or is suspected of having been exposed to ionizing radiation of at least 1 Gy.

14. The method of claim 13, wherein the subject has been or is suspected of having been exposed to ionizing radiation of at least 2 Gy.

15. A method of determining an absorbed dose of ionizing radiation in a subject who has been or is suspected of having been exposed to ionizing radiation, comprising:
a) obtaining a biological sample from the subject;
b) measuring levels of one or more inflammasome biomarkers selected from the group consisting of NLRP3, ASC, HMGB1, IL-1β, IL-6, IL-8, sICAM, and sVCAM in the biological sample to obtain an expression profile; and
c) determining absorbed radiation dose from the expression profile.

16. The method of claim 15, wherein the level is a protein level.

17. The method of claim 15, wherein the level is an mRNA level.

18. The method of claim 15, wherein measuring comprises ELISA, immunoassay, western blot, or dot blot, immunoprecipitation, RT-PCR, nucleic acid hybridization, or a combination thereof.

19. The method of claim 15, wherein the expression profile comprises a dose dependent increase in one or more levels of NLRP3, ASC, HMGB1, IL-1β, IL-6, IL-8, sICAM, and sVCAM.

20. The method of claim 19, wherein the expression profile comprises a dose dependent increase in one or more levels of NLRP3, HMGB1, IL-1β, IL-6, and/or sICAM.

21. The method of claim 15, wherein the measuring occurs 8 hours after exposure.

22. The method of claim 15, wherein the measuring occurs 24 hours after exposure.

23. The method of claim 15, wherein the measuring occurs 1 to 14 days after exposure.

24. The method of claim 15, wherein the biological sample comprises blood.

25. The method of claim 15, further comprising treating the subject for radiation exposure.

26. The method of claim 15, wherein the subject is a human.

27. The method of claim 15, wherein the subject has been or is suspected of having been exposed to ionizing radiation of at least 1 Gy.

28. The method of claim 27, wherein the subject has been or is suspected of having been exposed to ionizing radiation of at least 2 Gy.

29. The method of claim 28, wherein the subject has been or is suspected of having been exposed to ionizing radiation of at least 5 Gy.

30. The method of claim 29, wherein the subject has been or is suspected of having been exposed to ionizing radiation of at least 10 Gy.

31. The method of claim 30, wherein the subject has been or is suspected of having been exposed to ionizing radiation of at least 15 Gy.

32. The method of claim 31, wherein the subject has been or is suspected of having been exposed to ionizing radiation of at least 20 Gy.

33. A device configured to perform the method of any of claims 1-32.

34. The device of claim 33, comprising a platform configured to rapidly detect an inflammasome protein or mRNA from a blood drop sample.

35. The device of claim 34, wherein the platform is a portable miniature platform.

36. A radiation biodosimeter panel comprising a combination of NLRP3, ASC, HMGB1, IL-1β, IL-6, IL-8, sICAM, and sVCAM inflammasome biomarkers.

37. A method of detecting whether a subject has been exposed to ionizing radiation, comprising:
a) obtaining a biological sample from the subject; and
b) measuring a protein and/or mRNA level of one or more inflammasome biomarker selected from the group consisting of NLRP3, ASC, HMGB1, IL-1β, IL-6, IL-8, sICAM, and sVCAM in the biological sample, wherein increased levels as compared to a non-irradiated control indicate the subject has been exposed to ionizing radiation.

38. The method of claim 37, wherein measuring comprises ELISA, immunoassays, immunostaining and fluorescence assays, western blot, or dot blot, immunoprecipitation, RT-PCR, nucleic acid hybridization, or a combination thereof.

39. The method of claim 37, wherein increased levels of HMGB1 and/or sICAM-1 as compared to the control indicates the subject has been exposed to ionizing radiation.

40. The method of claim 37, wherein increased levels of three or more of the inflammasome biomarkers as compared to the control indicates the subject has been exposed to ionizing radiation.

41. The method of claim 37, wherein the measuring occurs 8 hours after exposure.

42. The method of claim 37, wherein the measuring occurs 24 hours after exposure.

43. The method of claim 37, wherein the measuring occurs 1 to 14 days after exposure.

44. The method of claim 37, wherein the biological sample comprises blood.

45. The method of claim 37, further comprising treating the subject for ionizing radiation exposure.

46. The method of claim 37, wherein the subject is a human.

47. The method of claim 37, wherein the subject has been or is suspected of having been exposed to ionizing radiation of at least 1 Gy.

48. The method of claim 46, wherein the subject has been or is suspected of having been exposed to ionizing radiation of at least 2 Gy.

49. The method of claim 47, wherein the subject has been or is suspected of having been exposed to ionizing radiation of at least 5 Gy.

50. The method of claim 47, wherein the subject has been or is suspected of having been exposed to ionizing radiation of from at least 10 Gy to at least 20 Gy.
